SEQUENCE LISTING (22 sequences)

SEQ ID NO: 1 - DNA, 11 bp, Artificial Sequence
  Description of Artificial Sequence Synthetic oligonucleotide
tctgtcnttct

SEQ ID NO: 2 - DNA, 11 bp, Artificial Sequence
  Description of Artificial Sequence Synthetic oligonucleotide
tctgacnttct

SEQ ID NO: 3 - DNA, 11 bp, Artificial Sequence
  Description of Artificial Sequence Synthetic oligonucleotide
tctgtcnttct

SEQ ID NO: 4 - DNA, 11 bp, Artificial Sequence
  Description of Artificial Sequence Synthetic oligonucleotide
tctgtngttct

SEQ ID NO: 5 - DNA, 11 bp, Artificial Sequence
  Description of Artificial Sequence Synthetic oligonucleotide
tctgtngttct

SEQ ID NO: 6 - DNA, 11 bp, Artificial Sequence
  Description of Artificial Sequence Synthetic oligonucleotide
tctgtngttct

SEQ ID NO: 7 - DNA, 11 bp, Artificial Sequence
  Description of Artificial Sequence Synthetic oligonucleotide
ctgtcnttctc

SEQ ID NO: 8 - DNA, 11 bp, Artificial Sequence
  Description of Artificial Sequence Synthetic oligonucleotide
ctgtcnttctc

SEQ ID NO: 9 - DNA, 11 bp, Artificial Sequence
  Description of Artificial Sequence Synthetic oligonucleotide
ctgtngttctc

SEQ ID NO: 10 - DNA, 11 bp, Artificial Sequence
  Description of Artificial Sequence Synthetic oligonucleotide
ctgtngttctc

SEQ ID NO: 11 - DNA, 11 bp, Artificial Sequence
  Description of Artificial Sequence Synthetic oligonucleotide
ctgtngttctc

SEQ ID NO: 12 - DNA, 11 bp, Artificial Sequence
  Description of Artificial Sequence Synthetic oligonucleotide
tcntcnttctg

SEQ ID NO: 13 - DNA, 11 bp, Artificial Sequence
  Description of Artificial Sequence Synthetic oligonucleotide
tcntcnttctg

SEQ ID NO: 14 - DNA, 11 bp, Artificial Sequence
  Description of Artificial Sequence Synthetic oligonucleotide
tngtngttctg

SEQ ID NO: 15 - DNA, 11 bp, Artificial Sequence
  Description of Artificial Sequence Synthetic oligonucleotide
tngtngttctg

SEQ ID NO: 16 - DNA, 11 bp, Artificial Sequence
  Description of Artificial Sequence Synthetic oligonucleotide
tngtngttctg

SEQ ID NO: 17 - DNA, 18 bp, Artificial Sequence
  Description of Artificial Sequence Synthetic oligonucleotide
ctatctgacgttctctgt

SEQ ID NO: 18 - DNA, 9 bp, Artificial Sequence
  Description of Artificial Sequence Synthetic oligonucleotide
tcntcnttg

SEQ ID NO: 19 - DNA, 8 bp, Artificial Sequence
  Description of Artificial Sequence Synthetic oligonucleotide
tcntcntt

SEQ ID NO: 20 - DNA, 8 bp, Artificial Sequence
  Description of Artificial Sequence Synthetic oligonucleotide
tcntcntt

SEQ ID NO: 21 - DNA, 67 bp, Artificial Sequence
  Description of Artificial Sequence Synthetic oligonucleotide
nnnnnnnnnnnnnnnnnnnnnnnnnnnnnnnnnnnnnnnnnnnnnnnnnnnnnnnnnnnnnnnnnnn

SEQ ID NO: 22 - DNA, 18 bp, Artificial Sequence
  Description of Artificial Sequence Synthetic oligonucleotide
ctatctcaccttctctgt

(19) United States
(12) Patent Application Publication
(10) Pub. No.: US 2011/0123540 A1
(43) Pub. Date: May 26, 2011

(54) POTENT MUCOSAL IMMUNE RESPONSE INDUCED BY MODIFIED IMMUNOMODULATORY OLIGONUCLEOTIDES

(75) Inventors: Daqing Wang, Bedford, MA (US); Ekambar Kandimalla, Southboro, MA (US); Sudhir Agrawal, Shrewsbury, MA (US); Fu-Gang Zhu, Bedford, MA (US)

(73) Assignee: Hybridon, Inc.

(21) Appl. No.: 12/952,868

(22) Filed: Nov. 23, 2010

Related U.S. Application Data
(63) Continuation of application No. 11/060,228, filed on Feb. 17, 2005, now abandoned.
(60) Provisional application No. 60/627,263, filed on Nov. 12, 2004.
(60) Provisional application No. 60/613,786, filed on Sep. 28, 2004.
(60) Provisional application No. 60/546,147, filed on Feb. 20, 2004.

(51) Int. Cl. A61K 39/395; A61K 39/00; A61K 39/35; A61K 39/39; A61P 37/02

(52) U.S. Cl. 424/141.1; 424/184.1; 424/130.1; 424/275.1

(57) ABSTRACT

The invention relates to the therapeutic use of immunostimulatory oligonucleotides and/or immunomers on mucosal innate immunity as well as adjuvant activity using ovalbumin (OVA) as an antigen through administration to the mucosal lining.

### RELATED APPLICATIONS

This application is a continuation of U.S. application Ser. No. 11/060,228, filed on Feb. 17, 2005 and claims the benefit of U.S. Provisional Application Ser. No. 60/627,263, filed on Nov. 12, 2004, and U.S. Provisional Application Ser. No. 60/613,786, filed on Sep. 28, 2004, and U.S. Provisional Application Ser. No. 60/546,147, filed on Feb. 20, 2004, the contents of which are incorporated by reference in their entirety.

### BACKGROUND OF THE INVENTION

1. Field of the Invention

The invention relates to applications using immunomers for inducing mucosal immune responses.

2. Summary of the Related Art

Recently, several researchers have demonstrated the validity of the use of oligonucleotides as immunostimulatory agents in immunotherapy applications. The observation that phosphodiester and phosphorothioate oligonucleotides can induce immune stimulation has created interest in developing these compounds as a therapeutic tool. These efforts have focused on phosphorothioate oligonucleotides containing the natural dinucleotide CpG. Kuramoto et al., Jpn. J. Cancer Res. 83:1128-1131 (1992) teaches that phosphodiester oligonucleotides containing a palindrome that includes a CpG dinucleotide can induce interferon-alpha and gamma synthesis and enhance natural killer activity. Krieg et al., Nature 371:546-549 (1995) discloses that phosphorothioate CpG-containing oligonucleotides are immunostimulatory. Liang et al., J. Clin. Invest. 98:1119-1129 (1996) discloses that such oligonucleotides activate human B cells. Moldoveanu et al., Vaccine 16:1216-124 (1998) teaches that CpG-containing phosphorothioate oligonucleotides enhance immune response against influenza virus. McCluskie and Davis, J. Immunol. 161:4463-4466 (1998) teaches that CpG-containing oligonucleotides act as potent adjuvants, enhancing immune response against hepatitis B surface antigen.

One response that CpG-containing oligonucleotides may modulate is asthma. An allergic asthma response is characterized by activation of T-helper type 2 (Th2) lymphocytes. The responses induced by Th2 lymphocytes play a major role in the pathogenesis and propagation of allergic inflammation in asthma. The Th2 cytokine IL-5 increases the generation and survival of eosinophils, leading to increased airway eosinophilia. Other Th2 cytokines (IL-4, IL-5, IL-9, and IL-13) also play critical roles in allergic inflammation by inducing production of allergen-specific IgE, mast-cell proliferation, endothelial-cell adhesion-molecule expression, and airway hyper-responsiveness. Corticosteroids are currently the most widely used treatment for allergic asthma. Steroid treatment is effective only in minimizing the manifestations of inflammation, however, but does not cure the disease. Continuous therapy is required to prevent the progression of allergic asthma.

These reports make clear that there remains a need to be able to enhance and modify the immune response caused by immunostimulatory oligonucleotides. However, the use of conventional CpG-containing DNAs for oral or intragastric administration is limited because of its rapid degradation in gastrointestinal environment. Thus, there remains a need for CpG DNAs (or other CpG analogue/motifs) with greater stability in the gastrointestinal environment to induce mucosal immune responses.

### BRIEF SUMMARY OF THE INVENTION

The invention provides compositions and methods for inducing mucosal immune responses. In a first aspect, the invention provides a method for generating a mucosal immune response in a vertebrate. In one embodiment of this aspect of the invention the method comprises administering an immunomer to the mucosal lining of the vertebrate.

In a second aspect, the invention provides a method for therapeutically treating a vertebrate having a disease or disorder. In one embodiment of this aspect of the invention the method comprises administering an immunomer to the mucosal lining of the vertebrate.

In a third aspect, the invention provides a method for modulating a mucosal immune response in a vertebrate. In one embodiment of this aspect of the invention the method comprises administering an immunomer to the mucosal lining of the vertebrate.

### BRIEF DESCRIPTION OF THE DRAWINGS

FIG. 1 shows MIP-13 (A), IP-10 (B) production in stomach extracts and IL-12 (C) levels in intestinal extracts four hrs post CpG* IMO or CpG DNA intragastric (i.g.) administration.

FIG. 2 shows serum IL-12 and MCP-1 levels at 4 hrs post CpG* IMO or CpG DNA i.g administration.

FIG. 3 shows serum MIP-β levels at 4 hrs post CpG* IMO or CpG DNA i.g administration.

FIG. 4 shows serum IL-12 level at 4 hrs post CpG* IMO or CpG DNA i.g administration.

FIG. 5 shows serum OVA specific IgG1 and IgG2a levels after i.g. administration of OVA mixed with CpG* IMO or CpG.

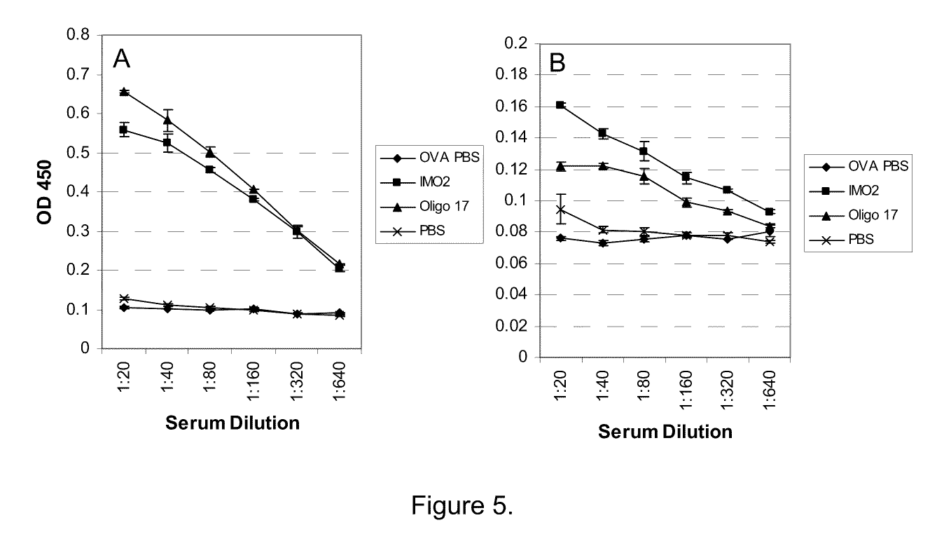

FIG. 6 shows local IgA level in intestinal washing after i.g. administration.

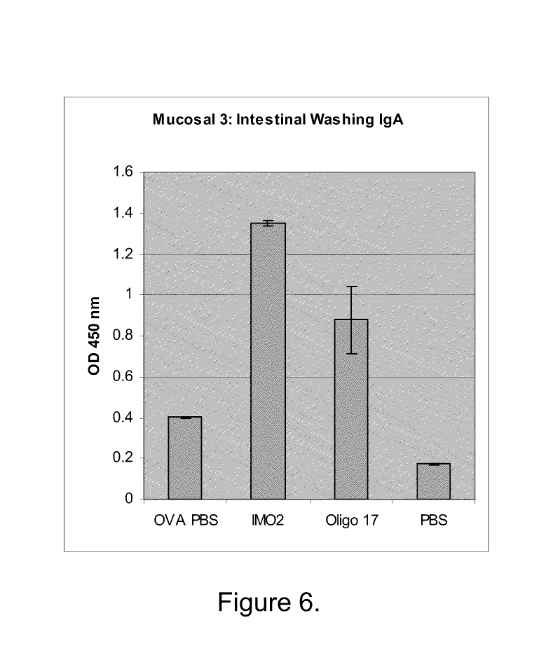

FIG. 7 shows anti-OVA IgG2a and IgG1 serum levels on day 42 post i.g. administration.

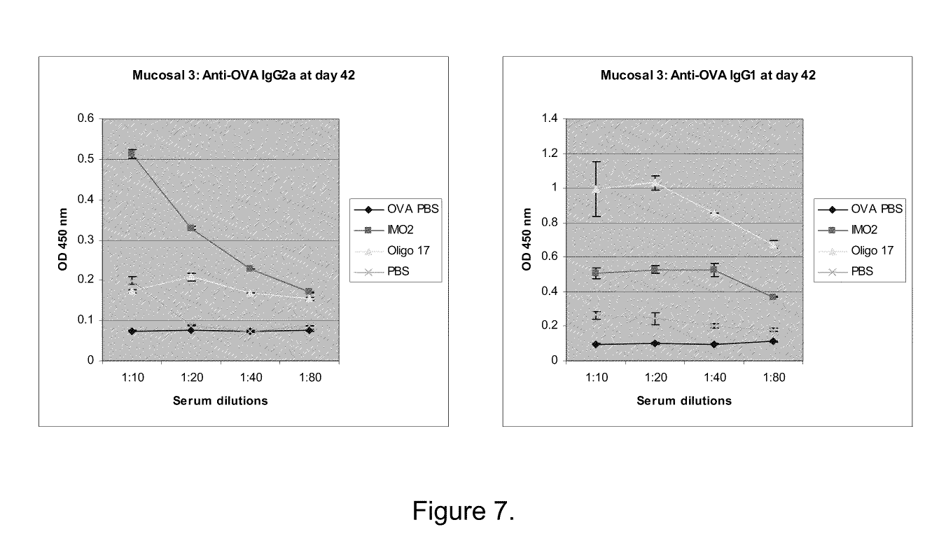

FIG. 8 shows anti-OVA IgG2a and IgG1 serum levels on day 35 post intrarectal (i.r.) administration.

FIG. 9 depicts the treatment protocol for IMO mediated intragastric vaccination and tumor challenge.

FIG. 10 shows IFN-γ secretion by T-cells in orally vaccinated mice.

FIG. 11 shows serum anti-OVA IgG2a and IgG1 responses following intragastric immunization with OVA mixed with IMO 2 or oligonucleotide 17 as mucosal adjuvants.

FIG. 12 shows anti-OVA IgA levels in intestinal washings and serum washings.

FIG. 13 shows induced immuno-protective effects against OVA-positive tumor challenge in mice i.g immunized with OVA mixed with IMO 2 or oligonucleotide 17.

FIG. 14 shows dose-dependent responses to mucosal immunization.

FIG. 15 depicts the OVA-specific IgG2a and IgG1 responses for a time course study of mucosal immune responses.

FIG. 16 depicts the treatment protocol for IMO challenged mice compared to mice challenged with budenoside.

FIG. 17 shows the effects of intranasal (i.n.) and s.c. administration of IMO on cytokine/chemokine levels in OVA-sensitized mice lung.

FIG. 18 shows the effects of i.n. and s.c. administration of IMO on serum antibody levels in OVA-sensitized mice.

FIG. 19 shows the effects of i.n. and s.c. administration of IMO on lung inflammation in OVA-sensitized mice.

FIG. 20 shows the effect of oral administration of IMO on serum Ig levels in OVA-sensitized and challenged mice.

FIG. 21 shows the effect of oral administration of IMO on BALF Ig levels in OVA-sensitized and challenged mice.

FIG. 22 shows the effect of oral administration of IMO on lung inflammation in OVA-sensitized mice.

FIG. 23 depicts the treatment protocol for intragastric vaccination.

FIG. 24 shows that t-cells specifically respond to OVA257-264 collected from mesenteric lymph nodes and spleens.

FIG. 25 shows that OVA mixed with 2048 induced stronger Th1 type responses, showing higher serum OVA-specific IgG2a (A), and suppressed (lower) OVA-specific IgG1 (B).

FIG. 26 shows that IMO-mediated intragastic OVA vaccinations induced local OVA-specific secreting IgA in intestinal washing.

FIG. 27 depicts the treatment protocol for intragastric vaccination.

FIG. 28 shows that the level of OVA-specific IgG2a at day 42 started to increase in OVA-2048 group.

FIG. 29 depicts the treatment protocol for intragastric vaccination and OVA-specific humoral responses.

FIG. 30 shows that persistent presence of OVA-2048 is needed for maintaining of Th1 dominated statue in OVA-2048 group.

FIG. 31 depicts the treatment protocol for intragastric vaccination and OVA-specific humoral responses.

FIG. 32 shows that OVA-2048 induced stronger Th1-type responses.

FIG. 33 depicts the treatment protocol for intragastric vaccination tumor challenge study.

FIG. 34 shows that both OVA-1182 and OVA-2048 elicited antigen-specific tumor rejection, however, different immune response profiles were elicited by OVA in 2048 or 1182 and may result in different immune protection against OVA positive EG-7 tumor cell challenge.

### DETAILED DESCRIPTION OF THE PREFERRED EMBODIMENTS

The invention relates to the therapeutic use of oligonucleotides as immunostimulatory agents for immunotherapy applications. Specifically, the invention relates to the therapeutic use of immunostimulatory oligonucleotides and/or immunomers on mucosal innate immunity as well as adjuvant activity using ovalbumin (OVA) as an antigen through oral, intragastric, intranasal, intratracheal, intravaginal and intrarectal administration. The issued patents, patent applications, and references that are cited herein are hereby incorporated by reference to the same extent as if each was specifically and individually indicated to be incorporated by reference. In the event of inconsistencies between any teaching of any reference cited herein and the present specification, the latter shall prevail for purposes of the invention.

The invention provides methods for enhancing and modifying the immune response caused by immunostimulatory compounds used for immunotherapy applications such as, but not limited to, treatment of cancer, autoimmune disorders, inflammatory bowel syndrome, ulcerated colitits, Crohn's disease, asthma, respiratory allergies, food allergies, and bacteria, parasitic, and viral infections in adult and pediatric human and veterinary applications. The invention further provides compounds having optimal levels of immunostimulatory effect for immunotherapy and methods for making and using such compounds. In addition, immunomers of the invention are useful as adjuvants in combination with DNA vaccines, antibodies, antigens, proteins, peptides, allergens, chemotherapeutic agents, and antisense oligonucleotides.

In a first aspect, the invention provides a method for generating a mucosal immune response in a vertebrate. In one embodiment of this aspect of the invention the method comprises administering an immunomer to the mucosal lining of the vertebrate.

For purposes of the invention, the term “CpG DNA” means an immunostimulotory oligonucleotide which contains a naturally occurring CpG dinucleotide or an immunostimulatory analog thereof. Preferred analogs include, without limitation, C*pG, CpG*, and C*pG*, wherein C is cytidine or 2′-deoxycytidine, C* is 2′-deoxythymidine, arabinocytidine, 1-(2′-deoxy-β-D-ribofuranosyl)-2-oxo-7-deaza-8-methyl-purine, 2′-deoxy-2′-substituted arabinocytidine, 2′-O-substituted arabinocytidine, 2′-deoxy-5-hydroxycytidine, 2′-deoxy-N4-alkyl-cytidine, 2′-deoxy-4-thiouridine or other non-natural pyrimidine nucleoside, G is guanosine or 2′-deoxyguanosine, G* is 2′-deoxy-7-deazaguanosine, 2′-deoxy-6-thioguanosine, 2′-deoxyinosine, arabinoguanosine, 2′-deoxy-2′ substituted-arabinoguanosine, 2′-O-substituted-arabinoguanosine, or other non-natural purine nucleoside, and p is an internucleoside linkage selected from the group consisting of phosphodiester, phosphorothioate, and phosphorodithioate. The term “oligonucleotide” refers to a polynucleoside formed from a plurality of linked nucleoside units. Such oligonucleotides can be obtained from existing nucleic acid sources, including genomic or cDNA, but are preferably produced by synthetic methods. In preferred embodiments each nucleoside unit includes a heterocyclic base and a pentofuranosyl, ribose, 2′-deoxyribose, trehalose, arabinose, 2′-deoxy-2′-substituted arabinose, 2′-O-substituted arabinose or hexose sugar group, or combinations thereof. The nucleoside residues can be coupled to each other by any of the numerous known internucleoside linkages. Such internucleoside linkages include, without limitation, phosphodiester, phosphorothioate, phosphorodithioate, alkylphosphonate, alkylphosphonothioate, phosphotriester, phosphoramidate, siloxane, carbonate, carboalkoxy, acetamidate, carbamate, morpholino, borano, thioether, bridged phosphoramidate, bridged methylene phosphonate, bridged phosphorothioate, and sulfone internucleoside linkages. The term “oligonucleotide” also encompasses polynucleosides having one or more stereospecific internucleoside linkage (e.g., (R_{P})— or (Sp)-phosphorothioate, alkylphosphonate, or phosphotriester linkages). As used herein, the terms “oligonucleotide” and “dinucleotide” are expressly intended to include polynucleosides and dinucleosides having any such internucleoside linkage, whether or not the linkage comprises a phosphate group. In certain preferred embodiments, these internucleoside linkages may be phosphodiester, phosphorothioate, or phosphorodithioate linkages, or combinations thereof.

In some embodiments, the oligonucleotides each have from about 3 to about 35 nucleoside residues, preferably from about 4 to about 30 nucleoside residues, more preferably from about 4 to about 20 nucleoside residues. In some embodiments, the oligonucleotides have from about 5 to about 18, or from about 5 to about 14, nucleoside residues. As used herein, the term “about” implies that the exact number is not critical. Thus, the number of nucleoside residues in the oligonucleotides is not critical, and oligonucleotides having one or two fewer nucleoside residues, or from one to several additional nucleoside residues are contemplated as equivalents of each of the embodiments described above. In some embodiments, one or more of the oligonucleotides have 11 nucleotides. Non-limiting examples of some nucleic acid molecules of the invention are presented in Tables 1A and 1B.

TABLE 1A

| Oligo or | |
|---|---|
| ImmunomerNo. | Sequences (5′-3′) |
| 1 | 5′-TCTGTCG₁TTCT-X-TCTTG₁CTGTCT-5′ |
| 2 | 5′-TCTGACG₁TTCT-X-TCTTG₁CAGTCT-5′ |
| 3 | 5′-TCTGTCG₂TTCT-X-TCTTG₂CTGTCT-5′ |
| 4 | 5′-TCTGTC₁GTTCT-X-TCTTGC₁TGTCT-5′ |
| 5 | 5′-TCTGTC₂GTTCT-X-TCTTGC₂TGTCT-5′ |
| 6 | 5′-TCTGTC₃GTTCT-X-TCTTGC₃TGTCT-5′ |
| 7 | 5′-CTGTCG₁TTCTC-X-CTCTTG₁CTGTC-5′ |
| 8 | 5′-CTGTCG₂TTCTC-X-CTCTTG₂CTGTC-5′ |
| 9 | 5′-CTGTC₁GTTCTC-X-CTCTTGC₁TGTC-5′ |
| 10 | 5′-CTGTC₂GTTCTC-X-CTCTTGC₂TGTC-5′ |
| 11 | 5′-CTGTC₃GTTCTC-X-CTCTTGC₃TGTC-5′ |
| 12 | 5′-TCG₁TCG₁TTCTG-X-GTCTTG₁CTG₁CT-5′ |
| 13 | 5′-TCG₂TCG₂TTCTG-X-GTCTTG₂CTG₂CT-5′ |
| 14 | 5′-TC₁GTC₁GTTCTG-X-GTCTTGC₁TGC₁T-5′ |
| 15 | 5′-TC₂GTC₂GTTCTG-X-GTCTTGC₂TGC₂T-5′ |
| 16 | 5′-TC₃GTC₃GTTCTG-X-GTCTTGC₃TGC₃T-5′ |
| 17 | 5′-CTATCTGACGTTCTCTGT-3′ |
| 18 | 5′-TCG₁TCG₁TTG-X-GTTG₁CTG₁CT |
| 19 | 5′-TCG₁TCG₁TT-YYY-GTCTCGAGAC |
| 20 | 5′-TCG₁TCG₁TT-YYY-GUCUCGAGAC |

* G₁ = 2′-deoxy-7-deazaguanosine; G₂ = arabinoguanosine.
C₁ = 2′-deoxycytidine, 1-(2′-deoxy-β-D-ribofuranosyl)-2-oxo-7-deaza-8-methylpurine; C₂ = arabinocytidine; C₃ = 2′-deoxy-5-hydroxycytidine.
X = Glycerol linker. Can also be C2-C18 alkyl linker, ethylene glycol linker, polyethylene glycol linker, branched alkyl linker.
Y = 1,3-Propanediol linker
A/U/C/G = 2′-O-methylribonucleosides

TABLE 1B

| Oligo or | | |
|---|---|---|
| ImmunomerNo. | Sequences (5′-3′) | Modifications |
| 16 | 5′-Nₙ N₁C¹G¹N₁C²G² N₁Nₙ-X- Nₙ N₁ | C¹, C², C³, and C⁴ are independently 2′- |
| | G³C³ N₁G⁴C⁴ N₁ Nₙ-5′ | deoxycytidine, 1-(2′-deoxy-β-D-ribofuranosyl)- |
| | | 2-oxo-7-deaza-8-methylpurine, arabinocytidine; |
| | | or 2′-deoxy-5-hydroxycytidine. |
| | | G¹, G², G³, and G⁴ are independently 2′-deoxy- |
| | | 7-deazaguanosine; arabinoguanosine; 2′- |
| | | deoxyinosine |

N₁ and Nn, independent at each occurrence, is preferably a naturally occurring or a synthetic nucleoside, wherein n is a number from 0 to 30
X = Glycerol linker. Can also be C2-C18 alkyl linker, ethylene glycol linker, polyethylene glycol linker, branched alkyl linker.

For purposes of the invention, the term “immunomer” refers to any compound comprising at least two oligonucleotides linked at their 3′ ends or internucleoside linkages, or functionalized nucleobase or sugar directly or via a non-nucleotidic linker, at least one of the oligonucleotides (in the context of the immunomer) being an immunostimulatory oligonucleotide and having an accessible 5′ end, wherein the compound induces an immune response when administered to a vertebrate. In some embodiments, the vertebrate is a mammal, including a human.

In some embodiments, the immunomer comprises two or more immunostimulatory oligonucleotides, (in the context of the immunomer) which may be the same or different. Preferably, each such immunostimulatory oligonucleotide has at least one accessible 5′ end.

Various embodiments of the invention provide an immunostimulatory nucleic acid comprising at least two oligonucleotides. In this aspect, immunostimulatory nucleic acid comprises a structure as detailed in formula (I).

Domain A-Domain B-Domain C (I)

Domains may be from about 2 to about 12 nucleotides in length. Domain A may be 5′-3′ or 3′-5′ or 2′-5′ DNA, RNA, RNA-DNA, DNA-RNA having or not having a palindromic or self-complementary domain containing or not containing at least one dinucleotide selected from the group consisting of CpG, C*pG, CpG*, and C*pG*, as described above.

In certain embodiments, Domain A will have more than one dinucleotide selected from the group consisting of CpG, C*pG, CpG*, and C*pG* located in the 5′-end of the Domain A oligonucleotide.

Domain B is a linker joining Domains A and C that may be a 3′-5′ linkage, a 2′-5′ linkage, a 3′-3′ linkage, a phosphate group, a nucleoside, or a non-nucleoside linker that may be aliphatic, aromatic, aryl, cyclic, chiral, achiral, a peptide, a carbohydrate, a lipid, a fatty acid, C2-C18 alkyl linker, poly(ethylene glycol) linker, ethylene glycol linker, branched alkyl linker, 2′-5′ internucleoside linkage, glycerol, mono- tri- or hexapolyethylene glycol, or a heterocyclic moiety.

Domain C may be 5′-3′ or 3′-5′, 2′-5′ DNA, RNA, RNA-DNA, DNA-RNA Poly I-Poly C having or not having a palindromic or self-complementary sequence, which can or cannot have a dinucleotide selected from the group consisting of CpG, C*pG, CpG*, and C*pG*, as described above.

In certain embodiments, the invention provides “CpG” DNAs containing two distinct domains, namely stimulatory and structural domains, referred to as self-stabilized CpG DNAs. The stimulatory domain of CpG DNAs contained a naturally occurring CpG dinucleotide or an immunostimulatory analog thereof at the 5′-end. In the structural domain region, complementary sequences that formed 7, 11, 15, or 19 base-pair (bp) hairpin stem-loop structures were incorporated adjacent to the 3′-end of the stimulatory domain. In these embodiments, the immunostimulatory nucleic acid has a secondary structure at the 3′-end by way of hydrogen bonding with a complementary sequence. As used herein, the term “secondary structure” refers to intramolecular and intermolecular hydrogen bonding. Intramolecular hydrogen bonding results in the formation of a stem-loop structure. Intermolecular hydrogen bonding results in the formation of a duplexed nucleic acid molecule. These CpG DNAs were designed such that the stimulatory domain did not contain any structural motifs (base-pairing) and CpG stimulatory motifs may or may not be present in the structural domain. The nucleosides within the structural domain may contain sugar, phosphate and/or base modifications that improve stability of the hairpin stem-loop structures, which further improves the stability of the self-stabilized CpG DNAs against endonuclease digestion in the gastrointestinal environment.

For purposes of the invention, the term “oligonucleotide” refers to a polynucleoside formed from a plurality of linked nucleoside units. Such oligonucleotides can be obtained from existing nucleic acid sources, including genomic or cDNA, but are preferably produced by synthetic methods. In preferred embodiments each nucleoside unit includes a heterocyclic base and a pentofuranosyl, 2′-deoxypentfuranosyl, trehalose, arabinose, 2′-deoxy-2′-substituted arabinose, 2′-O-substituted arabinose or hexose sugar group. The nucleoside residues can be coupled to each other by any of the numerous known internucleoside linkages. Such internucleoside linkages include, without limitation, phosphodiester, phosphorothioate, phosphorodithioate, alkylphosphonate, alkylphosphonothioate, phosphotriester, phosphoramidate, siloxane, carbonate, carboalkoxy, acetamidate, carbamate, morpholino, borano, thioether, bridged phosphoramidate, bridged methylene phosphonate, bridged phosphorothioate, and sulfone internucleoside linkages. The term “oligonucleotide” also encompasses polynucleosides having one or more stereospecific internucleoside linkage (e.g., (RP) or (SP)-phosphorothioate, alkylphosphonate, or phosphotriester linkages). As used herein, the terms “oligonucleotide” and “dinucleotide” are expressly intended to include polynucleosides and dinucleosides having any such internucleoside linkage, whether or not the linkage comprises a phosphate group. In certain preferred embodiments, these internucleoside linkages may be phosphodiester, phosphorothioate, or phosphorodithioate linkages, or combinations thereof.

The term “oligonucleotide” also encompasses polynucleosides having additional substituents including, without limitation, protein groups, lipophilic groups, intercalating agents, diamines, folic acid, cholesterol and adamantane. The term “oligonucleotide” also encompasses any other nucleobase containing polymer, including, without limitation, peptide nucleic acids (PNA), peptide nucleic acids with phosphate groups (PHONA), locked nucleic acids (LNA), morpholino-backbone oligonucleotides, and oligonucleotides having backbone sections with alkyl linkers or amino linkers.

The oligonucleotides of the invention can include naturally occurring nucleosides, modified nucleosides, or mixtures thereof. As used herein, the term “modified nucleoside” is a nucleoside that includes a modified heterocyclic base, a modified sugar moiety, or a combination thereof. In some embodiments, the modified nucleoside is a non-natural pyrimidine or purine nucleoside, as herein described. In some embodiments, the modified nucleoside is a 2′-substituted ribonucleoside an arabinonucleoside or a 2′-deoxy-2′-substituted-arabinoside.

For purposes of the invention, the term “2′-substituted ribonucleoside” or “2′-substituted arabinoside” includes ribonucleosides or arabinonucleoside in which the hydroxyl group at the 2′ position of the pentose moiety is substituted to produce a 2′-substituted or 2′-O-substituted ribonucleoside. Preferably, such substitution is with a lower alkyl group containing 1-6 saturated or unsaturated carbon atoms, or with an aryl group having 6-10 carbon atoms, wherein such alkyl, or aryl group may be unsubstituted or may be substituted, e.g., with halo, hydroxy, trifluoromethyl, cyano, nitro, acyl, acyloxy, alkoxy, carboxyl, carboalkoxy, or amino groups. Examples of 2′-O-substituted ribonucleosides or 2′-β-substituted-arabinosides include, without limitation 2′-O-methylribonucleosides or 2′-β-methylarabinosides and 2′-O-methoxyethylribonucleosides or 2′-O-methoxyethylarabinosides.

The term “2′-substituted ribonucleoside” or “2′-substituted arabinoside” also includes ribonucleosides or arabinonucleosides in which the 2′-hydroxyl group is replaced with a lower alkyl group containing 1-6 saturated or unsaturated carbon atoms, or with an amino or halo group. Examples of such 2′-substituted ribonucleosides or 2′-substituted arabinosides include, without limitation, 2′-amino, 2′-fluoro, 2′-allyl, and 2′-propargyl ribonucleosides or arabinosides.

The term “oligonucleotide” includes hybrid and chimeric oligonucleotides. A “chimeric oligonucleotide” is an oligonucleotide having more than one type of internucleoside linkage. One preferred example of such a chimeric oligonucleotide is a chimeric oligonucleotide comprising a phosphorothioate, phosphodiester or phosphorodithioate region and non-ionic linkages such as alkylphosphonate or alkylphosphonothioate linkages (see e.g., Pederson et al. U.S. Pat. Nos. 5,635,377 and 5,366,878).

A “hybrid oligonucleotide” is an oligonucleotide having more than one type of nucleoside. One preferred example of such a hybrid oligonucleotide comprises a ribonucleotide or 2′ substituted ribonucleotide region, and a deoxyribonucleotide region (see, e.g., Metelev and Agrawal, U.S. Pat. Nos. 5,652,355, 6,346,614 and 6,143,881).

For purposes of the invention, the term “immunostimulatory oligonucleotide” refers to an oligonucleotide as described above that induces an immune response when administered to a vertebrate, such as a fish, bird, or mammal. As used herein, the term “mammal” includes, without limitation rats, mice, cats, dogs, horses, cattle, cows, pigs, rabbits, non-human primates, and humans. Preferably, the immunostimulatory oligonucleotide comprises at least one phosphodiester, phosphorothioate, methylphosphonate, or phosphordithioate internucleoside linkage.

The immunomers used in the methods according to the invention comprise at least two oligonucleotides linked directly or via a non-nucleotidic linker. For purposes of the invention, a “non-nucleotidic linker” is any moiety that can be linked to the oligonucleotides by way of covalent or non-covalent linkages. Preferably, the linker is C2-C18 alkyl linker, poly(ethylene glycol) linker, ethylene glycol linker, branched alkyl linker, 2′-5′ internucleoside linkage, or glycerol or a glycerol homolog of the formula HO—(CH₂)ₒ—CH(OH)—(CH₂)ₚ—OH, wherein o and p independently are integers from 1 to about 6, from 1 to about 4, or from 1 to about 3.

In the methods according to this invention, administration of immunomodulatory oligonucleotide and/or immunomer can be by any suitable route, including, without limitation, oral, intragastric, intranasal, intratracheal, intravaginal or intrarectal. Administration of the therapeutic compositions of immunomers can be carried out using known procedures at dosages and for periods of time effective to reduce symptoms or surrogate markers of the disease. When administered systemically, the therapeutic composition is preferably administered at a sufficient dosage to attain a blood level of immunomer from about 0.0001 micromolar to about 10 micromolar. For localized administration, much lower concentrations than this may be effective, and much higher concentrations may be tolerated. Preferably, a total dosage of immunomer ranges from about 0.001 mg per patient per day to about 200 mg per kg body weight per day. It may be desirable to administer simultaneously, or sequentially a therapeutically effective amount of one or more of the therapeutic compositions of the invention to an individual as a single treatment episode.

The immunostimulatory oligonucleotides and/or immunomers used in the methods according to the invention may conveniently be synthesized using an automated synthesizer and phosphoramidite approach. In some embodiments, the immunostimulatory oligonucleotides and/or immunomers are synthesized by a linear synthesis approach. As used herein, the term “linear synthesis” refers to a synthesis that starts at one end of the immunomer and progresses linearly to the other end. Linear synthesis permits incorporation of either identical or un-identical (in terms of length, base composition and/or chemical modifications incorporated) monomeric units into the immunostimulatory oligonucleotides and/or immunomers.

An alternative mode of synthesis for immunomers is “parallel synthesis”, in which synthesis proceeds outward from a central linker moiety. A solid support attached linker can be used for parallel synthesis, as is described in U.S. Pat. No. 5,912,332. Alternatively, a universal solid support, such as phosphate attached to controlled pore glass support, can be used.

Parallel synthesis of immunomers has several advantages over linear synthesis: (1) parallel synthesis permits the incorporation of identical monomeric units; (2) unlike in linear synthesis, both (or all) the monomeric units are synthesized at the same time, thereby the number of synthetic steps and the time required for the synthesis is the same as that of a monomeric unit; and (3) the reduction in synthetic steps improves purity and yield of the final immunomer product.

At the end of the synthesis by either linear synthesis or parallel synthesis protocols, the immunostimulatory oligonucleotides or immunomers used in the method according to the invention may conveniently be deprotected with concentrated ammonia solution or as recommended by the phosphoramidite supplier, if a modified nucleoside is incorporated. The product immunostimulatory oligonucleotides and/or immunomer is preferably purified by reversed phase HPLC, detritylated, desalted and dialyzed.

The invention further provides pharmaceutical formulations comprising any of the immunostimulatory oligonucleotides disclosed herein either alone or in combination and a physiologically acceptable carrier. As used herein, the term “physiologically acceptable” refers to a material that does not interfere with the effectiveness of the immunostimulatory oligonucleotide and is compatible with a biological system such as a cell, cell culture, tissue, or organism. Preferably, the biological system is a living organism, such as a vertebrate.

As used herein, the term “carrier” encompasses any excipient, diluent, filler, salt, buffer, stabilizer, solubilizer, lipid, or other material well known in the art for use in pharmaceutical formulations. It will be understood that the characteristics of the carrier, excipient, or diluent will depend on the route of administration for a particular application. The preparation of pharmaceutically acceptable formulations containing these materials is described in, e.g., Remington's Pharmaceutical Sciences, 18th Edition, ed. A. Gennaro, Mack Publishing Co., Easton, Pa., 1990.

The invention provides methods for therapeutically treating a patient having a disease or disorder, such methods comprise administering to the mucosal lining, either by oral, intragastric, intranasal, intratracheal, intravaginal or intrarectal administration, of the patient an immunomer or immunomer conjugate according to the invention. The invention also provides methods for modulating an immune response in a vertebrate, such methods comprise administering to the mucosal lining, either by oral, intragastric, intranasal, intratracheal, intravaginal or intrarectal administration, of the vertebrate an immunomer or immunomer conjugate according to the invention. In various embodiments, the disease or disorder to be treated is cancer, an autoimmune disorder, inflammatory bowel syndrome, ulcerated colitis, Crohn's disease, airway inflammation, inflammatory disorders, allergy, asthma or a disease caused by a pathogen. Pathogens include bacteria, parasites, fungi, viruses, viroids and prions.

The immunomer conjugate comprises an immunomer, as described above, and an antigen conjugated to the immunomer at a position other than the accessible 5′ end. In some embodiments, the non-nucleotidic linker comprises an antigen, which is conjugated to the oligonucleotide. In some other embodiments, the antigen is conjugated to the oligonucleotide at a position other than its 3′ end. In some embodiments, the antigen produces a vaccine effect.

For purposes of the invention, the term “allergy” includes, without limitation, food allergies atopic dermatitis, allergic rhinitis (also known as hay fever), allergic conjunctivitis, urticaria (also known as hives), respiratory allergies and allergic reactions to other substances such as latex, medications and insect stings or problems commonly resulting from allergic rhinitis-sinusitis and otitis media. The term “airway inflammation” includes, without limitation, asthma. Specific examples of asthma include, but are not limited to, allergic asthma, non-allergic asthma, exercised-induced asthma, occupational asthma, and nocturnal asthma.

Allergic asthma is characterized by airway obstruction associated with allergies and triggered by substances called allergens. Triggers of allergic asthma include, but are not limited to, airborne pollens, molds, animal dander, house dust mites and cockroach droppings. Non-allergic asthma is caused by viral infections, certain medications or irritants found in the air, which aggravate the nose and airways. Triggers of non-allergic asthma include, but are not limited to, airborne particles (e.g., coal, chalk dust), air pollutants (e.g., tobacco smoke, wood smoke), strong odors or sprays (e.g., perfumes, household cleaners, cooking fumes, paints or varnishes), viral infections (e.g., colds, viral pneumonia, sinusitis, nasal polyps), aspirin-sensitivity, and gastroesophageal reflux disease (GERD). Exercise-induced asthma (EIA) is triggered by vigorous physical activity. Symptoms of EIA occur to varying degrees in a majority of asthma sufferers and are likely to be triggered as a result of breathing cold, dry air while exercising. Triggers of EIA include, but are not limited to, breathing airborne pollens during exercise, breathing air pollutants during exercise, exercising with viral respiratory tract infections and exercising in cold, dry air. Occupational asthma is directly related to inhaling irritants and other potentially harmful substances found in the workplace. Triggers of occupational asthma include, but are not limited to, fumes, chemicals, gases, resins, metals, dusts, vapors and insecticides.

Without wishing to be bound to any particular theory, decreased exposure to bacteria may be partially responsible for the increased incidence of, severity of, and mortality due to allergic diseases such as asthma, atopic dermatitis, and rhinitis in the developed countries. This hypothesis is supported by evidence that bacterial infections or products can inhibit the development of allergic disorders in experimental animal models and clinical studies. Bacterial DNA or synthetic oligodeoxynucleotides containing unmethylated CpG dinucleotides in certain sequence contexts (CpG DNA) potently stimulate innate immune responses and thereby acquired immunity. The immune response to CpG DNA includes activation of innate immune cells, proliferation of B cells, induction of Th1 cytokine secretion, and production of immunoglobulins (Ig). The activation of immune cells by CpG DNA occurs via Toll-like receptor 9 (TLR9), a molecular pattern recognition receptor. CpG DNAs induce strong Th1-dominant immune responses characterized by secretion of IL-12 and IFN-\tilde{γ}. Immunomers (IMO) alone or as allergen conjugates decrease production of IL-4, IL-5, and IgE and reduce eosinophilia in mouse models of allergic asthma. IMO compounds also effectively reverse established atopic eosinophilic airway disease by converting a Th2 response to a Th1 response.

OVA with alum is commonly used to establish a Th2-dominant immune response in various mouse and rat models. The Th2 immune response includes increased IL-4, IL-5, and IL-13 production, elevated serum levels of total and antigen-specific IgE, IgG1, and lower levels of IgG2a. IMO compounds prevent and reverse established Th2-dominant immune responses in mice. The co-administration of IMO compounds with OVA/alum to mice reduces IL-4, IL-5, and IL-13 production and induces IFN-γ production in spleen-cell cultures subjected to antigen re-stimulation. Furthermore, IMO compounds inhibit antigen-specific and total IgE and enhance IgG2a production in these mice.

Injection of OVA/alum and IMO compounds induces a lymphocyte antigen-recall response (Th1-type) in mice characterized by low levels of Th2-associated cytokines, IgE and IgG1, and high levels of Th1-associated cytokines and IgG2a. Co-administration of IMO compounds with other kinds of antigens, such as S. masoni egg and hen egg lysozyme, also result in reversal of the Th2-response to a Th1-dominant response in in vitro and in vivo studies. As described herein, IMO compounds effectively prevent development of a Th2 immune response and allow a strong Th1 response.

While Th2 cytokines trigger an Ig isotype switch towards production of IgE and IgG1, the Th1 cytokine IFN-γ induces production of IgG2a by B-lymphocytes. Mice injected with OVA/alum and IMO compounds produce lower levels of IL-4, IL-5, and IL-13 and higher levels of IFN-γ, accompanied by lower IgE and IgG1 and higher IgG2a levels, than mice injected with OVA/alum alone. This suggests the existence of a close link between Th1-cytokine induction and immunoglobulin isotype switch in mice that receive antigen and IMO compounds.

Serum antigen-specific and total IgE levels are significantly lower in mice receiving OVA/alum and IMO compounds than in mice receiving OVA/alum alone. In contrast, OVA-specific IgG1 levels are insignificantly changed and total IgG1 levels are only slightly decreased compared with mice injected with OVA/alum alone (data not shown). The different response may result from different mechanisms involved in the control of IgE and IgG1 class switch, though both isotypes are influenced by IL-4 and IL-13. For example, IL-6 promotes B lymphocytes to synthesize IgG1 in the presence of IL-4.

In any of the methods according to the invention, the immunomer or immunomer conjugate can be administered in combination with any other agent useful for treating the disease or condition that does not diminish the immunostimulatory effect of the immunomer. For purposes of this aspect of the invention, the term “in combination with” means in the course of treating the same disease in the same patient, and includes administering the immunomer and an agent in any order, including simultaneous administration, as well as any temporally spaced order, for example, from sequentially with one immediately following the other to up to several days apart. Such combination treatment may also include more than a single administration of the immunomer, and independently the agent. The administration of the immunomer and agent may be by the same or different routes.

In any of the methods according to the invention, the agent useful for treating the disease or condition includes, but is not limited to, antigen, allergen, or co-stimulatory molecules such as cytokines, chemokines, protein ligands, trans-activating factors, peptides and peptides comprising modified amino acids. Additionally, the agent can include DNA vectors encoding for antigen or allergen.

Oligonucleotides containing CpG motifs (CpG DNAs) activate innate immune system through TLR 9. As demonstrated herein, CpG oligonucleotides attached through 3′-3′-linkage, referred to as immunomers (IMO), are more potent inducers of TLR 9-mediated immune responses compared with conventional CpG DNAs. In the case of IMO, the presence of novel structure (absence of free 3′-end) attributes to greater stability in gastrointestinal tract providing an enhanced inducement of mucosal immunity through administration to the mucosal lining.

As shown in the examples, a single dose of IMO induced significantly higher levels of chemokines (MIP 1β, MCP1, and IP10) and cytokines (IL-12) locally (stomach and/or small intestine) and systemically (serum). On the contrary, under the same conditions and at the same dose, conventional CpG DNA oligonucleotides produced insignificant effects locally and systemically. Mice immunized intragastrically with OVA plus IMO at 1:1 ratio had significantly lower levels of anti-OVA IgG1 compared with mice similarly immunized with OVA plus CpG DNA. Mice immunized with OVA plus IMO showed significantly higher levels of OVA-specific IgG2a compared with mice immunized with OVA plus conventional CpG DNA. Under the same experimental conditions a non-CpG DNA had no effect on both local and systemic chemokine/cytokine secretion and immunoglobulin production. These results demonstrate that IMO containing novel structure are stable in GI track and induced potent mucosal immune responses compared with conventional CpG DNA. These results also demonstrate the mucosal Th1 adjuvant activity of IMO with vaccines and antigens through administration to the mucosal lining.

The invention provides a kit comprising a immunostimulatory oligonucleotides and/or immunomers, the latter comprising at least two oligonucleotides linked together, such that the immunomer has more than one accessible 5′ end, wherein at least one of the oligonucleotides is an immunostimulatory oligonucleotide. In another aspect, the kit comprises an immunostimulatory oligonucleotide and/or immunostimulatory oligonucleotide conjugate and/or immunomer or immunomer conjugate according to the invention and a physiologically acceptable carrier. The kit will generally also include a set of instructions for use.

The examples below are intended to further illustrate certain preferred embodiments of the invention, and are not intended to limit the scope of the invention.

### EXAMPLES

### Example 1

### Synthesis and Purification of IMO

CpG* DNA: (5′-TCTGACG*TTCT-X-TCTTG*CAGTCT-5′), wherein X and G* are glycerol linker and 2′-deoxy-7-deazaguanosine, respectively, a conventional CpG DNA (5′-CTATCTGACGTTCTCTGT-3′) and a non-CpG DNA: (5′-CTATCTCACCTTCTCTGT-3′) were synthesized, purified, and analyzed as previously described.

### Example 2

### Mice

Female C57BL/6 mice 5-8 weeks of age, were purchased from Jackson Laboratory (Bar Harbor, Me.). Mice were maintained in accordance with the Hybridon's IACUC approved animal protocols.

### Example 3

### Intragastric Administration

All mice were sedated lightly by isoflurane inhalation before administration of IMO. Three to 5 mice in each group received single 200 nl of PBS containing 5 mg/kg or 15 mg/kg IMO, CpG DNA or non-CpG DNA control through intragastric (i.g) with an 18-gauge feeding needle attached to a tuberculin syringe. Blood, stomach and small intestine were collected at different time points from 4-120 hr when mice were sacrificed.

For chicken egg ovalbumin (OVA, grade V, Sigma, St. Louis, Mo.) immunization group, 200 μl of PBS containing 100 ng of OVA and 100 ng IMO or CpG DNA were i.g administrated. Control mice were immunized with 100 ng OVA in 200 nl PBS or PBS only. All mice were boosted i.g at day 14. Blood and intestinal washings were collected on day 24 to 42 for determining OVA-specific antibody levels.

### Example 4

### Extraction of Chemokines from Tissues

Extraction of chemokines from the stomachs and small intestines was performed by using method described by Johansson et al with some modifications. Briefly, fresh organs collected at various time points were weighted, cut into small pieces and immediately frozen at −70° C. in PBS solution containing 2 mM phenylmethanesulfonyl fluoride (Sigma), 0.1 mg of soybean trypsin inhibitor (Sigma) per, and 0.05 M EDTA. The samples were thawed at room temperature and then permeabilized with saponin (Sigma) at a final concentration of 2% (wt/vol) in PBS at 4° C. overnight with continuing rotation. The tissue samples were then centrifuged at 16,000×g for 20 min, and the supernatants were analyzed by ELISA.

### Example 5

### Chemokine Quantification

Concentrations of macrophage inflammatory protein 113 (MIP-1β), monocyte chemotactic protein 1 (MCP-1) and interferon γ-induced protein 10 (IP-10) in the tissue extracts and serum samples were determined by using DuoSet ELISA development system (R&D, Minneapolis, Minn.) according to the manufacturer's recommendations.

### Example 6

### Assessment of Cytokine Levels

IL-12 levels from the tissue extracts and serum samples were determined by sandwich ELISA as described previously. Wells of ELISA plates (Costar, Corning, N.Y.) were coated with an antibody specific for mouse IL-12, after with 100 μl of serial diluted tissue extracts or serum were added to each well. IL-12 content was determined according to the manufacturer's instructions and expressed as pg/ml. All reagents, including cytokine antibodies and standards were purchased from PharMingen. (San Diego, Calif.).

### Example 7

### Antibody Determination

For the analysis of serum antibodies, 96 well plates were incubated at room temperature for 3 hours with OVA (grade V, Sigma, St. Louis, Mo.) at 2 μg/ml in phosphate buffered saline (PBS). The solid phase was incubated overnight at 4° C. with the intestinal washings or serum samples followed by an incubation with horseradish peroxidase (HRP)-conjugated goat anti-mouse IgG1, IgG2a or IgA (PharMingen, San Diego, Calif.). The binding of antibodies was measured as absorbance at 405 nm after reaction of the immune complexes with ABTS substrate (Zymed, San Francisco, Calif.).

### Example 8

### Histology

The stomachs, small intestines and mesentery lymph nodes collected at different time points after i.g. administration were fixed in 10% phosphate-buffered formalin (VWR, West Chester, Pa.) and then embedded in paraffin. Sections 5 μm thick were cut and stained with hematoxylin and eosin.

### Example 9

### Intrarectal Immunization

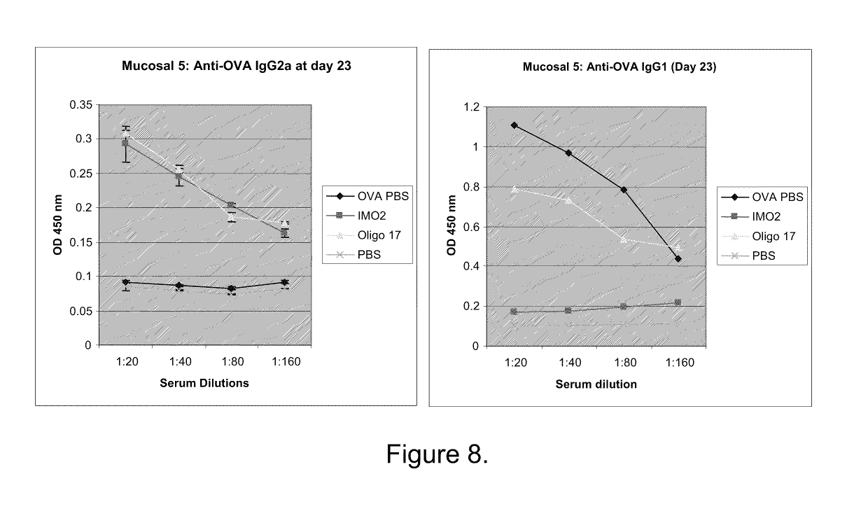

BALB/c female mice (n=5) were intrarectal administrated with 100 ug OVA protein mixed with 100 ul IMO in 50 ul PBS twice at day 1 and day 14. Mice administrated with 100 ug OVA in 50 ul PBS or 50 ul PBS only were used as control. Sera were taken at day 23 and OVA specific IgG1, IgG2a, IgA and IgE were tested by ELISA (see FIG. 8).

### Example 10

### Effects of CpG* IMO and Conventional CpG DNA on Mucosal and Systemic Innate Immunity

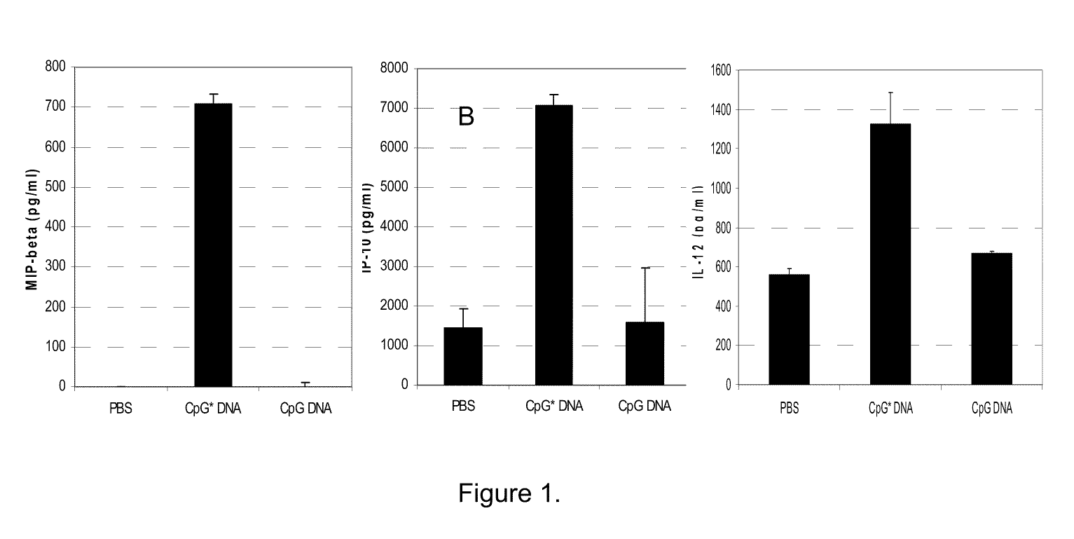

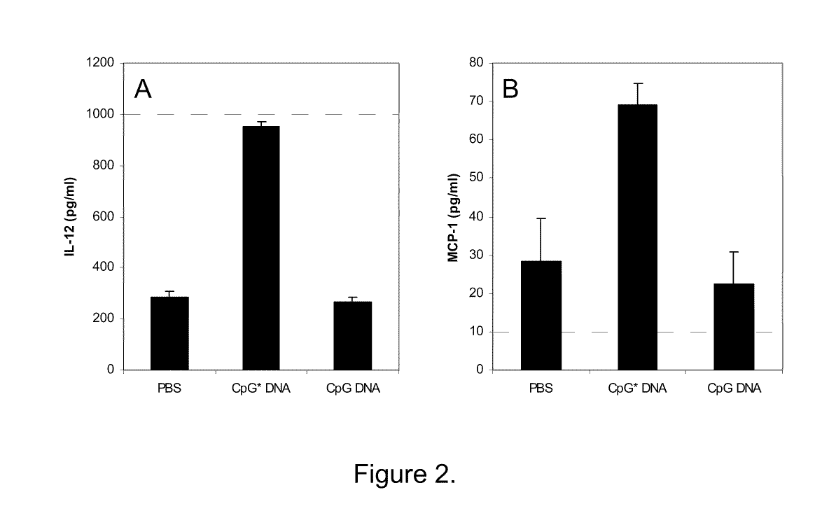

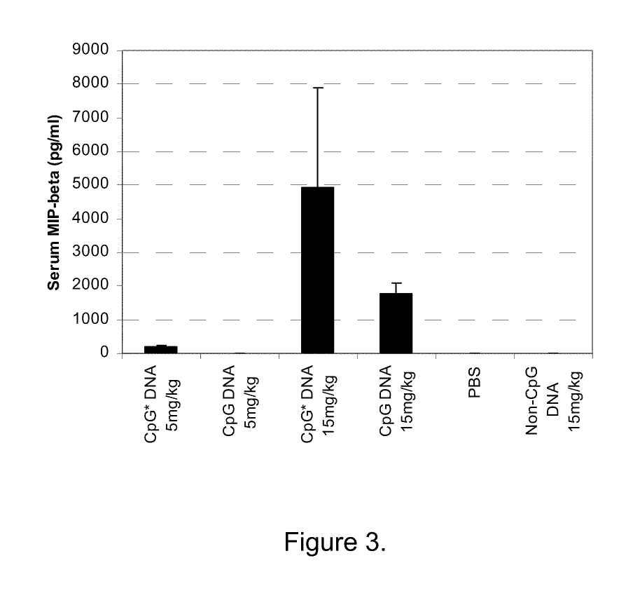

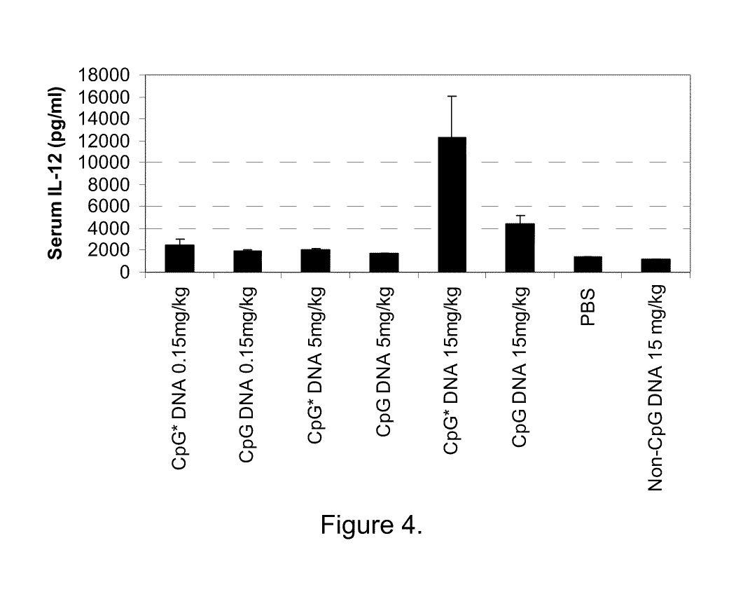

A single dose of 5 mg/kg CpG* IMO administrated i.g. produced higher levels of chemokines (MIP 1β, IP10 and MCP-1) and cytokine (IL-12) locally (stomach and/or intestine, FIG. 1) and systemically (serum, FIG. 2). Under the same conditions and dose, conventional CpG DNA produced insignificant effects (FIGS. 1 and 2). Compared with CpG* IMO, CpG DNA produced lower levels of serum MIP-13 (FIG. 3) and IL-12 (FIG. 4). A non-CpG DNA had no effect on local and systemic chemokine/cytokine secretion (FIG. 3, 4).

### Example 11

### CpG* IMO as a Vaccine Adjuvant

C57BL/6 female mice (n=5) were i.g. administrated with 100 μg OVA protein mixed with 100 μg IMO in 200 μl PBS twice at day 1 and day 14. Mice i.g. administrated with 100 μg OVA in 200 μl PBS or 200 μl PBS only were used as control. Sera were taken at day 23 and OVA specific IgG1 and IgG2a were determined by ELISA. Mice immunized with OVA plus CpG* IMO showed significantly higher levels of OVA-specific IgG2a and suppressed OVA-specific IgG1 production compared with mice immunized with OVA plus conventional CpG DNA.

These results clearly indicate that IMO containing novel structures and CpG* motif induced potent mucosal immune responses as a result of their higher stability in GI environment. Additionally, oral or intragastric administration of CpG* IMO induced potent mucosal Th1 adjuvant activity with OVA compared with conventional CpG DNA.

### Example 12

### IMO Mediated Intragastric Vaccination

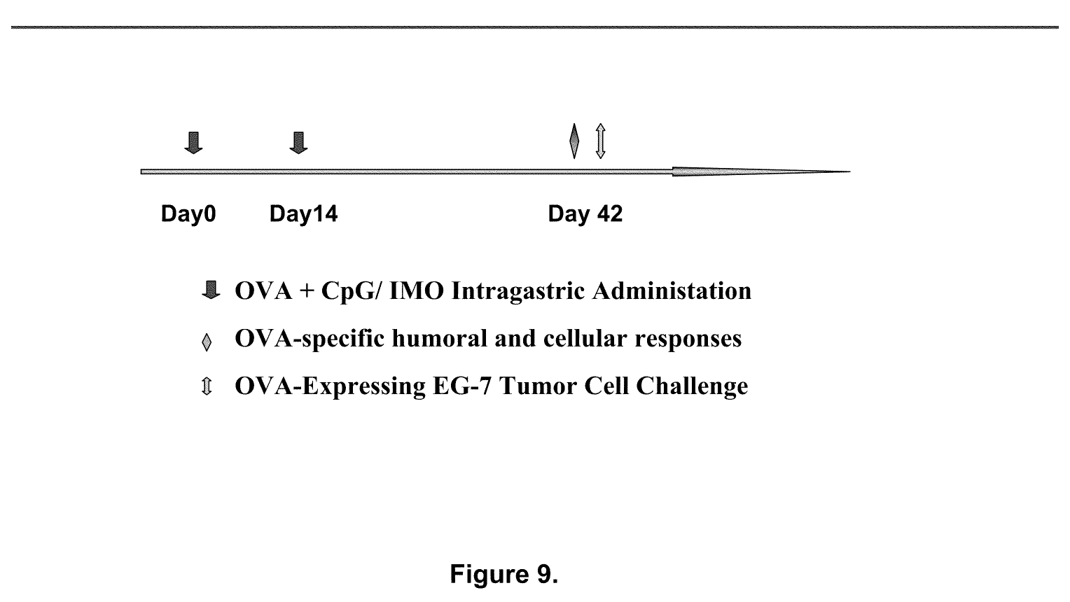

C57BL/6 female mice (n=10) were intragastrically (i.g) administrated with 25 mg/kg OVA protein alone or mixed with 15 mg/kg Oligo 17 or IMO 2 in 400 μl PBS on days 1 and 14. Three mice from each group were sacrificed on day 42. OVA-specific antibody and T cell responses were evaluated by ELISA and IFN-γ ELISPOT. Seven mice from each group were challenged with EG-7 tumor cells expressing OVA to determine whether oral administration of IMO would result in tumor rejection (see FIG. 9).

### Example 13

### IFN-γ Secretion by T-Cells in Orally Vaccinated Mice

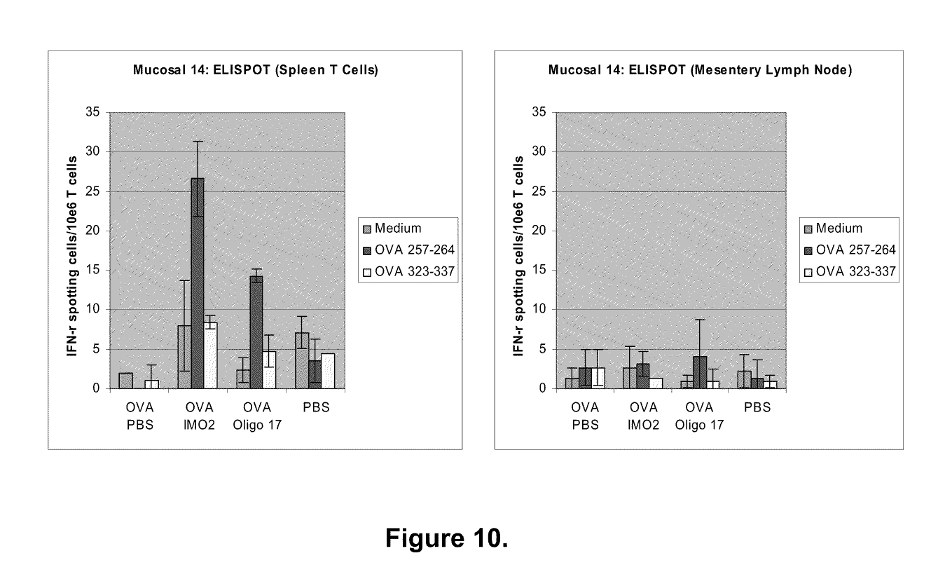

Spleens and mesenteric lymph nodes from immunized mice (n=3) (See Example 12) were collected on day 42. T cells were purified from pooled splenocytes and lymph nodes using T cell enrichment columns. 2.5×10⁵ T cells were stimulated with 2.5×10⁵ mitomycin C treated T cells and the immunodominant OVA₂₅₇₋₂₆₄ or OVA₃₂₃₋₃₃₇ pulsed syngeneic spleen cells for 24 hrs. T cells specifically responding to MHC class I restricted, OVA₂₅₇₋₂₆₄ restimulation were determined by IFN-γ ELISPOT analysis according to the manufacturer's directions (R&D Systems). Spots were enumerated electronically (Zellnet, New York, N.Y.). Both Oligo 17 and IMO 2 produced higher levels of IFN-γ secretion compared with OVA alone. FIG. 10 shows that oral administration of IMO 2 with OVA elicited stronger systemic H-2 kb restricted, OVA₂₅₇₋₂₆₄ specific T cell responses in splenocytes compared with Oligo 17.

### Example 14

### Serum Anti-OVA IgG2a and IgG1 Responses Following Intragastric Immunization with OVA Mixed with IMO 2 or Oligo 17 as Mucosal Adjuvants

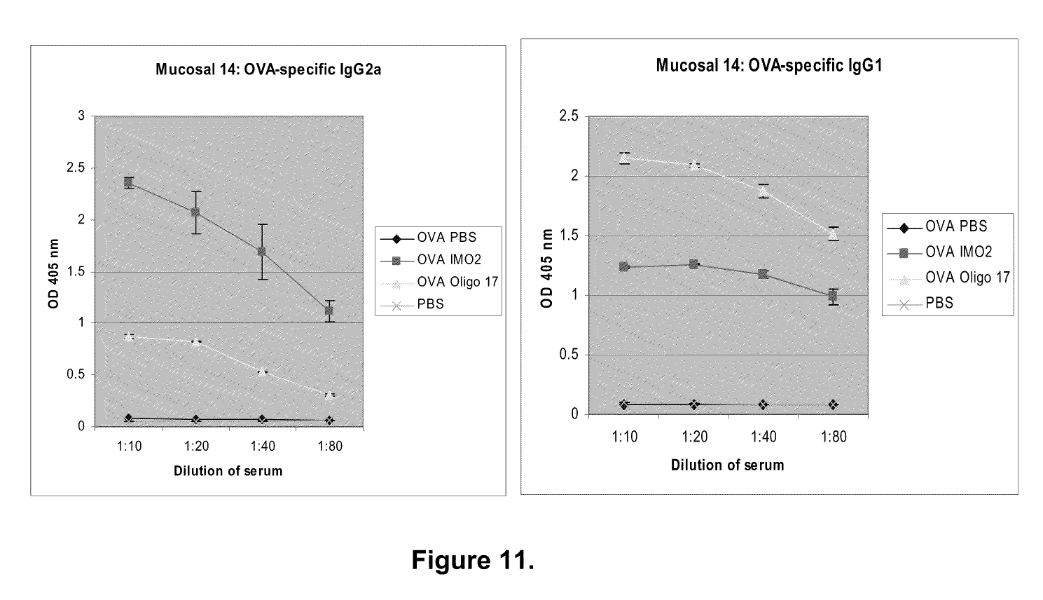

Serum samples collected on day 42 post original immunization (See Example 12) were evaluated for OVA-specific IgG2a (A) and IgG1 (B) by ELISA. Compared with Oligo 17, IMO 2 induced significantly higher serum OVA-specific IgG2a and suppressed anti-OVA IgG1. As shown in FIG. 11, the strong induction of H-2 kb restricted, OVA₂₅₇₋₂₆₄ specific CTL and OVA-specific IgG2a suggest that IMO 2 elicited potent Th1 immune responses.

### Example 15

### Anti-OVA IgA Levels in Intestinal Washings

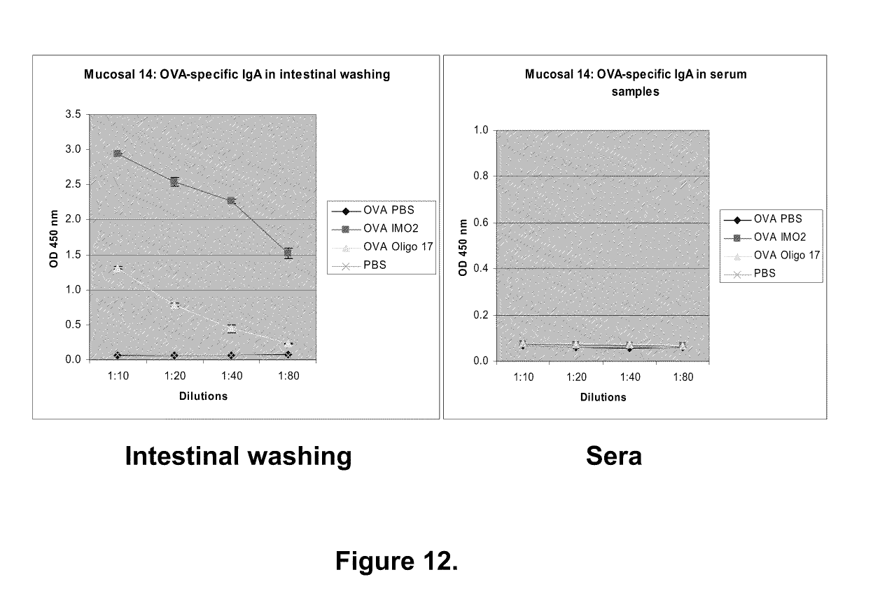

The intestinal washings collected on day 42 post original immunization were analyzed for OVA-specific IgA by ELISA. FIG. 12 shows that IMO 2 induced higher levels of OVA-specific IgA in intestinal tissue compared with Oligo 17.

### Example 16

### Mice i.g Immunized with OVA Mixed with IMO 2 or Oligo 17 Induced Immuno-Protective Effects Against OVA-Positive Tumor Challenge

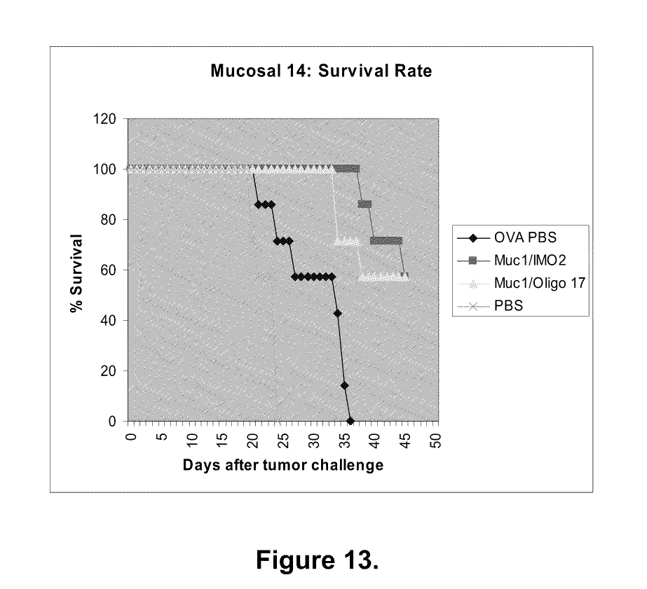

C57BL/6 mice were i.g vaccinated as described in Example 12. The immunized mice (n=7) were challenged with 1.5×10⁶ EG-7 cells expressing OVA. 100% of mice in control groups i.g administrated with PBS or OVA protein alone died of tumor burden with an average survival time of 26.4 days. About 43% mice in OVA mixed with IMO 2 or Oligo 17 i.g immunized mice were tumor-free for more than 55 days. FIG. 13 shows that the survival times of the mice that died of tumor in OVA mixed IMO 2 or Oligo 17 i.g group were significantly prolonged to 43 and 38.8 days respectively.

### Example 17

### Dose-Dependent Responses to Mucosal Immunization

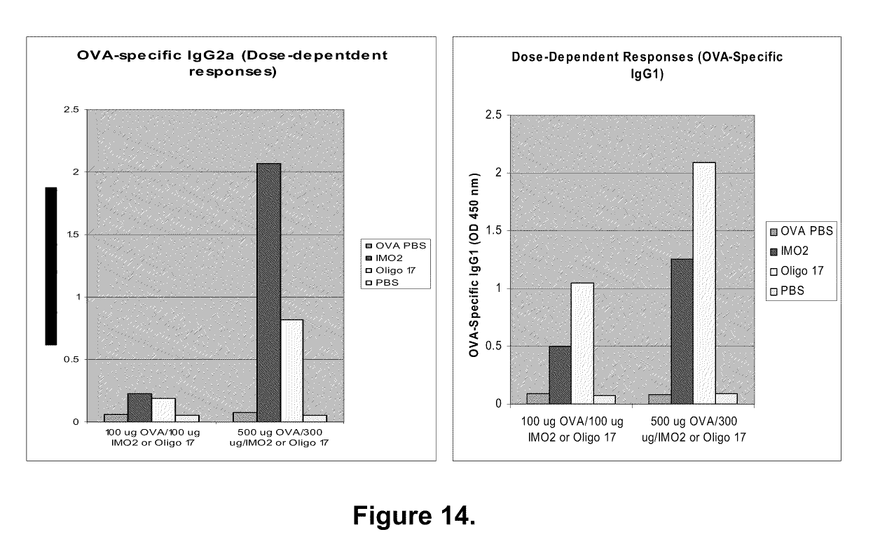

C57BL/6 mice were i.g. administered 100 mg OVA mixed with 100 mg IMO 2 or Oligo 17, or 500 mg OVA mixed with 300 mg IMO 2 or Oligo 17 at day 1 and day 14. Serum samples collected at day 42 and analyzed by ELISA for anti-OVA IgG2a and anti-OVA IgG1. FIG. 14 shows that IMO 2 induced higher anti-OVA IgG2a and lower IgG1 in both lower and higher dose i.g. administrations.

### Example 18

### Time Course Study of Mucosal Immune Responses

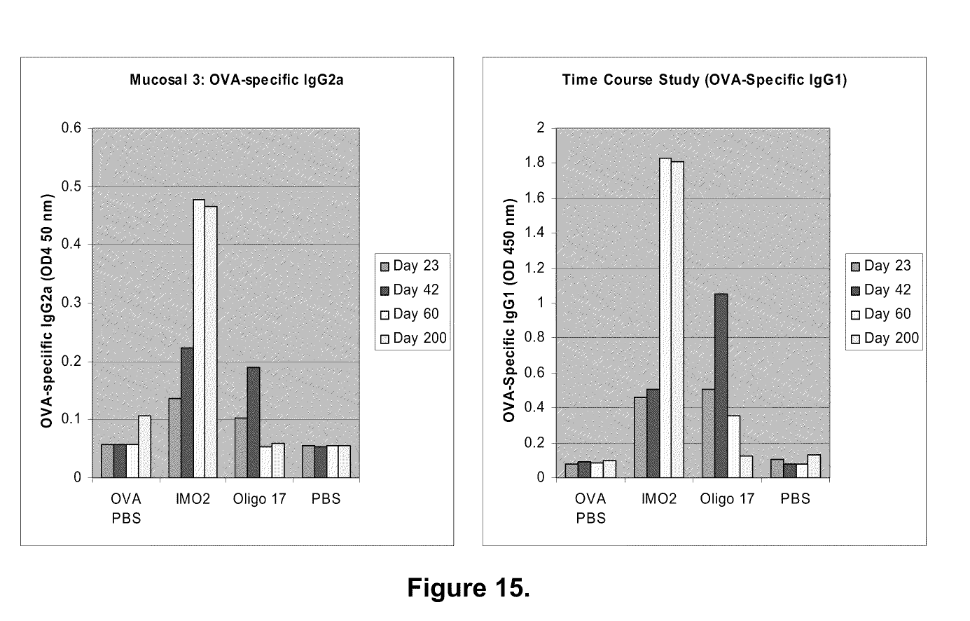

C57BL/c mice were vaccinated i.g. twice with 100 mg OVA only, 100 mg OVA mixed with 100 mg IMO 2 or Oligo 17 at day 1 and day 14. Serum samples were collected at days 23, 42, 60 and 200. OVA-specific IgG1 and IgG2a were determined by ELISA. FIG. 15 shows that IMO2/OVA produced higher levels of both IgG1 and IgG2a than either OVA alone or Oligo 17/OVA, peaking at day 60 and remaining elevated at day 200.

### Example 19

### Response Parameters to Treatment Protocol

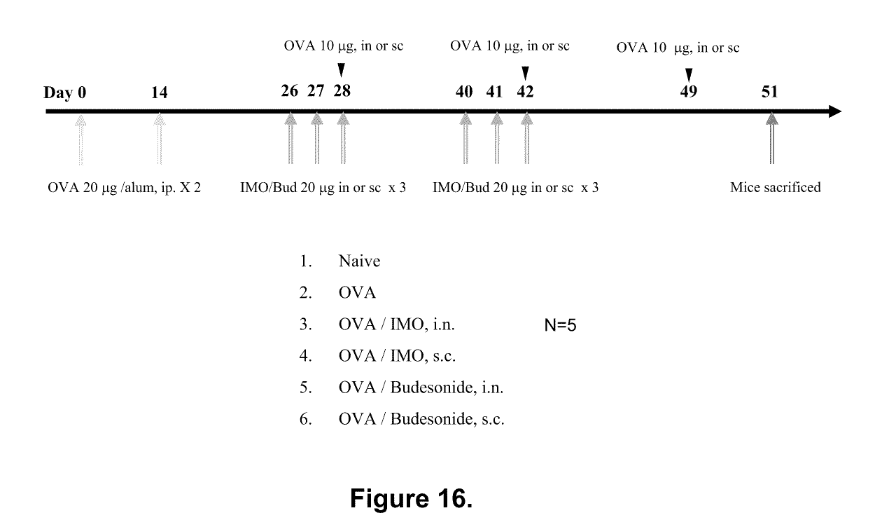

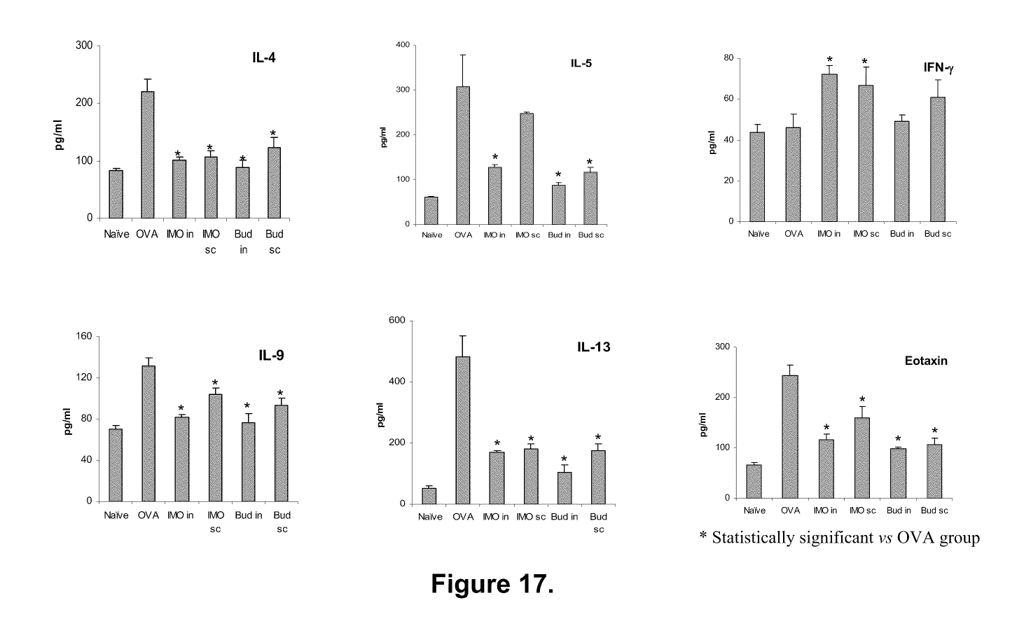

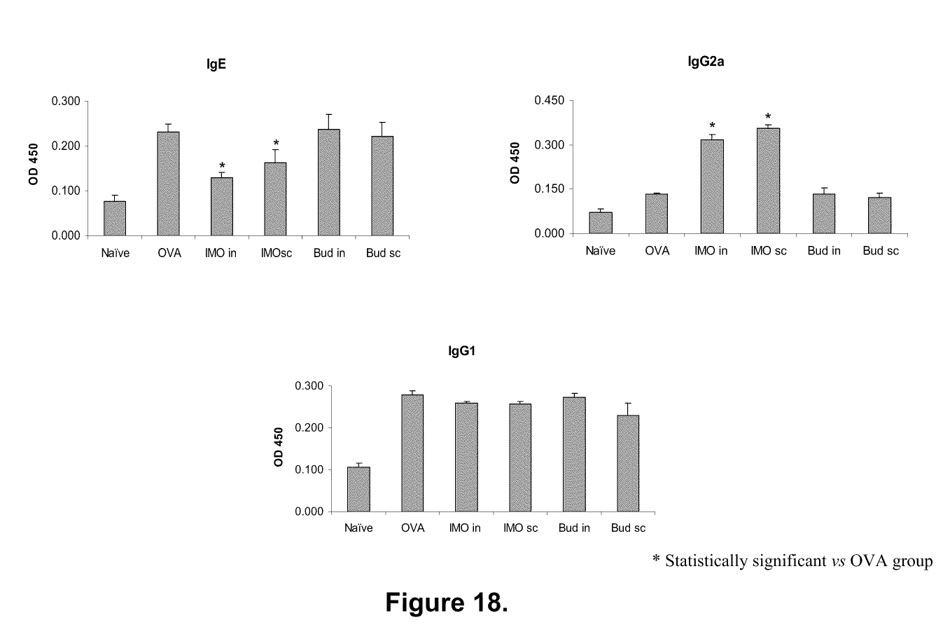

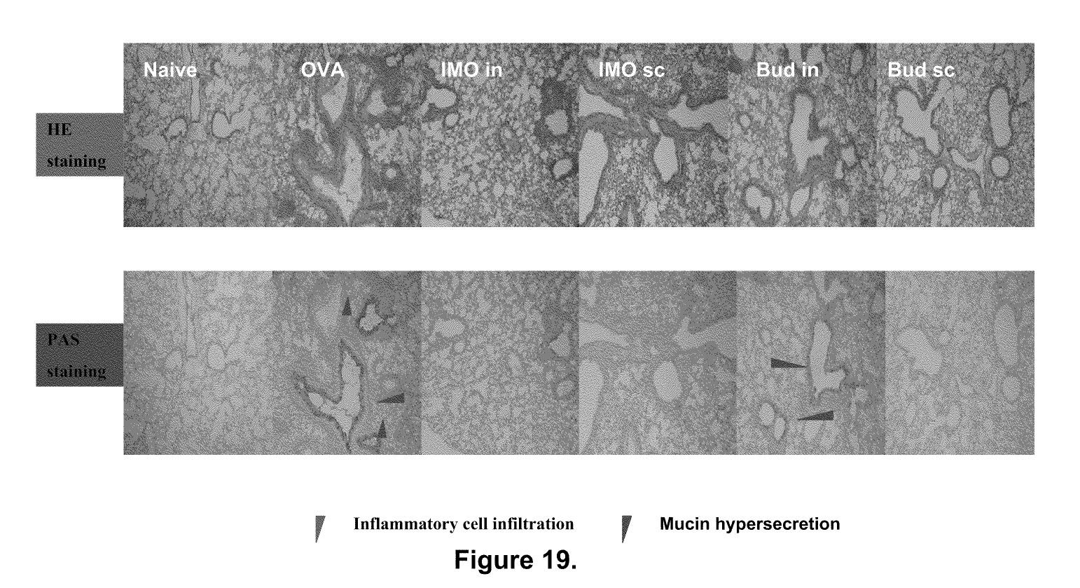

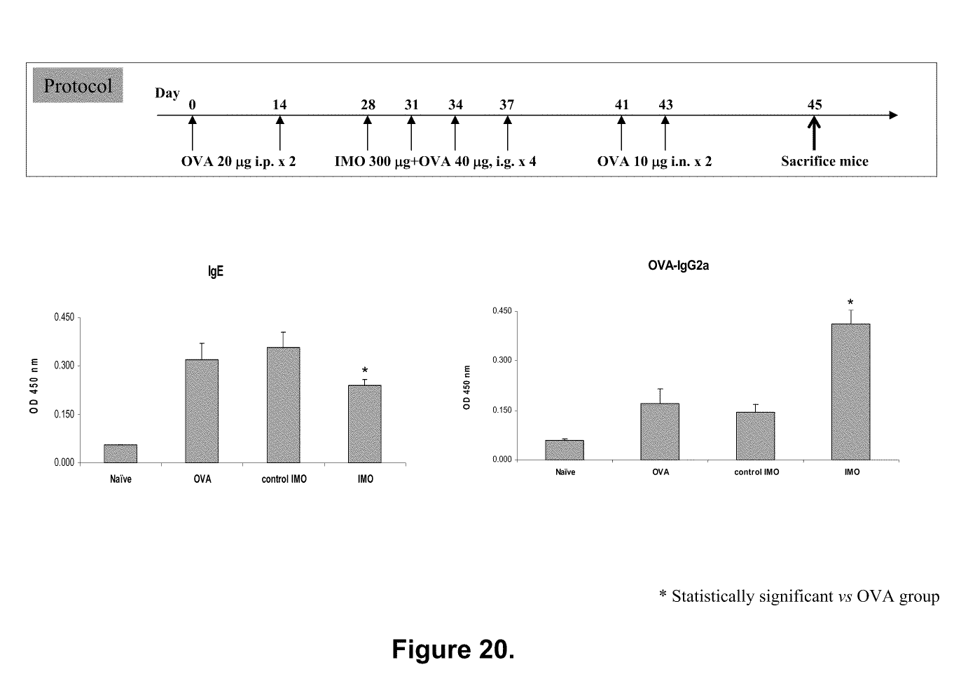

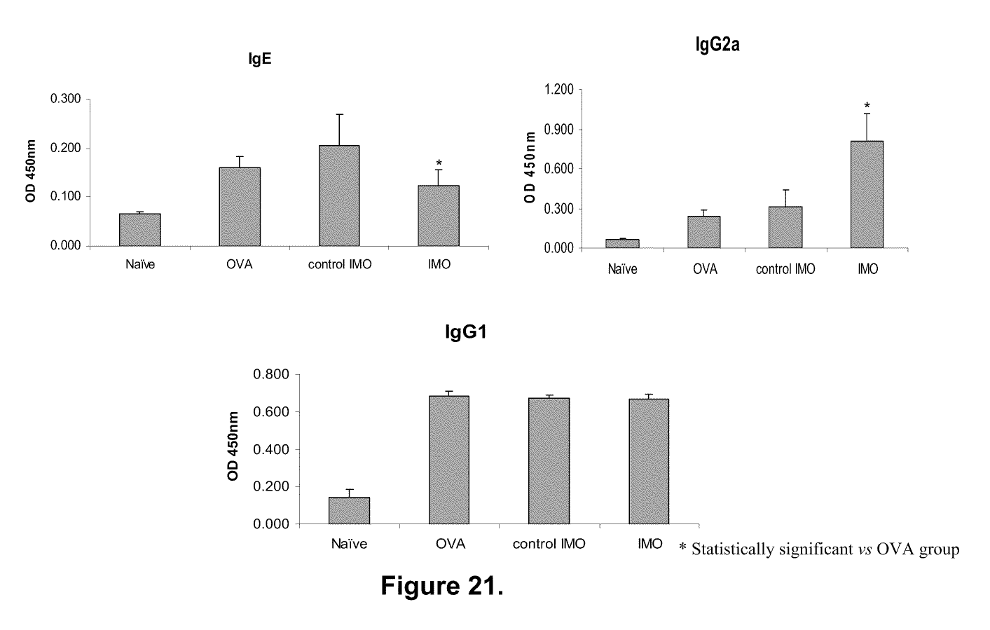

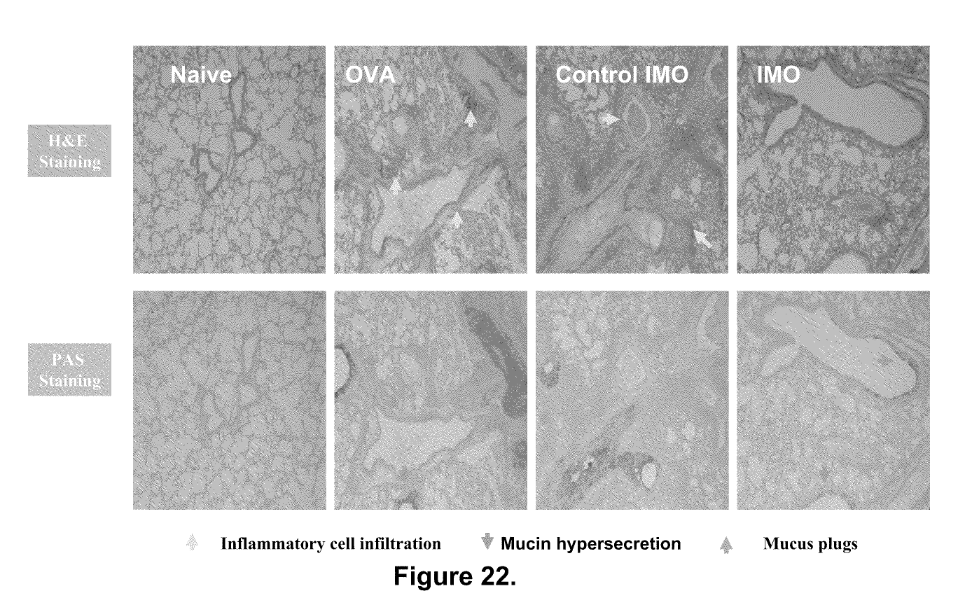

Treatment protocol as shown in FIG. 16. FIGS. 17-22 show the results. Either intranasal (i.n.) or subcutaneous (s.c.) administration of IMO suppressed Th2 cytokines and induced Th1 cytokine, IFN-γ, production in the lungs of OVA-sensitized and challenged mice. IMO, but not budenoside, suppressed serum IgE and increased serum IgG2a in OVA-sensitized and challenged mice. IMO, but not budenoside, reduced inflammatory cell infiltration and mucus hypersecretion in the lungs of OVA-sensitized and challenged mice. Orally administered IMO, but not control IMO, suppressed serum OVA-specific IgE and induced OVA-specific IgG2a, as well as reduced lung inflammation and mucin hypersecretion. In summary, IMO containing synthetic motifs are potent Th1 immunostimulators and inhibitors of Th2 cytokine production. IMO containing CpG* dinucleotide administered intranasally or subcutaneously reversed Th2 responses in a murine model of allergic asthma with ovalbumin (OVA). Orally administered IMO effectively reversed Th2 responses in the lung compared with control IMO.

### Example 20

### Intragastric Vaccination

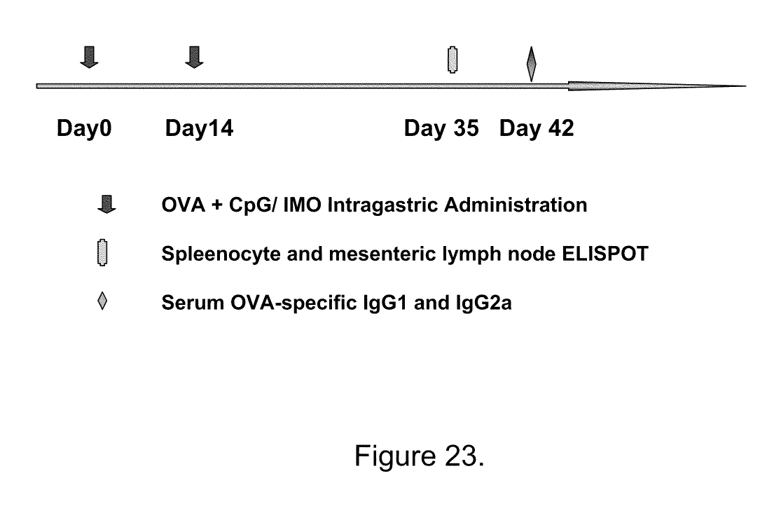

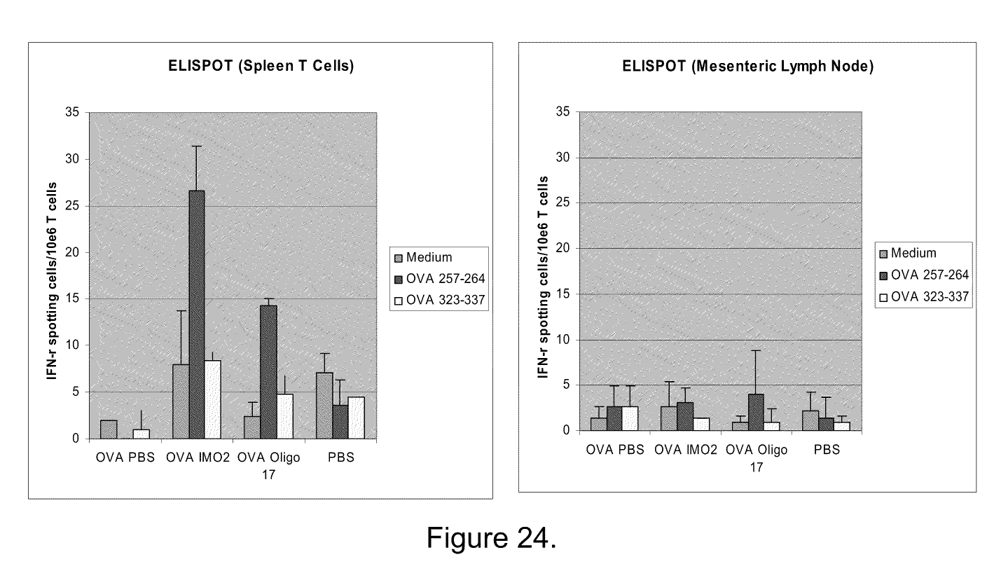

Female C57BL/6 mice were sedated lightly by isoflurane inhalation before oligo administration. 15 mg/kg IMO 2048 or 1182 or CpG DNA mixed with 25 mg/kg chicken ovalbumin (OVA) (grade V, Sigma, St. Louis, Mo.) in 400 μl of PBS was administered via intragastric administration (i.g.) at days 1 and 14 and control mice were immunized with 25 mg/kg OVA in 400-μl PBS (FIG. 23 shows the treatment protocol). FIGS. 24 through 26 show the results.

To determine the OVA-specific IFN-γ-secreting CTL responses three mice from each group were sacrificed at day 35 to 42. and t-cells from splenocytes and mesenteric lymph nodes in each group were purified using t-cell enrichment columns (R&D systems, Minneapolis, Minn.). Purified T cells (2.5×105) were stimulated with 2.5×10⁵ mitomycin C (50 μg/ml, Sigma, St. Louis, Mo.) and inactivated APCs pulsed with OVA257-264 or OVA323-337 peptides for 24 hrs. The number of CTL secreting IFN-γ in response to stimulation with the OVA257-264 peptide was determined by ELISPOT (R&D System). Serum samples (n=7) were taken on day 42 and OVA-specific serum antibody responses were evaluated on day 42 by ELISA. FIG. 24 shows that t-cells specifically respond to OVA257-264 collected from mesenteric lymph nodes and spleens as determined by IFN-γ ELISPOT.

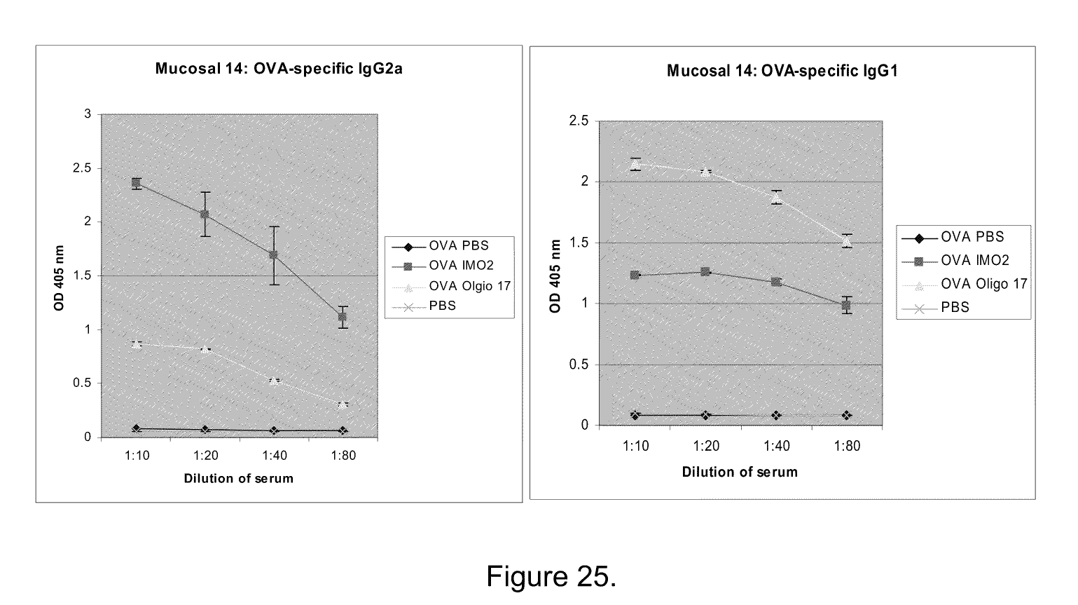

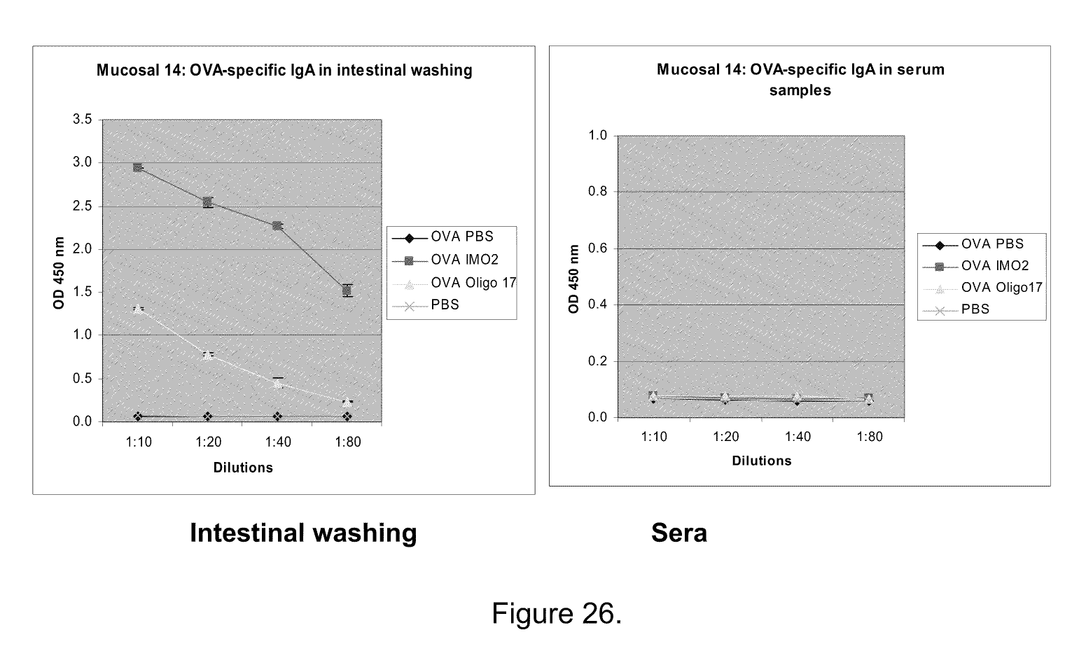

For OVA-specific antibody determination, 96-well plates were incubated at room temperature for 3 hr with OVA at 2 μg/ml in PBS. The solid phase was incubated overnight at 4° C. with the intestinal washings or serum samples collected at day 35 to 42 after the first immunization, followed by incubation with horseradish peroxidase (HRP)-conjugated goat anti-mouse IgG1, IgG2a, or IgA (PharMingen, San Diego, Calif.). The binding of antibodies was measured as absorbance at 450 nm after reaction of the immune complexes with ABTS substrate (Zymed, San Francisco, Calif.). As shown in FIG. 25, OVA mixed with 2048 induced stronger Th1 type responses, showing higher serum OVA-specific IgG2a (A), and suppressed (lower) OVA-specific IgG1 (B). Additionally, as shown if FIG. 26, IMO-mediated intragastic OVA vaccinations induced local OVA-specific secreting IgA in intestinal washing.

### Example 21

### IMO-Mediated Intragastric Vaccination Time-Course Study

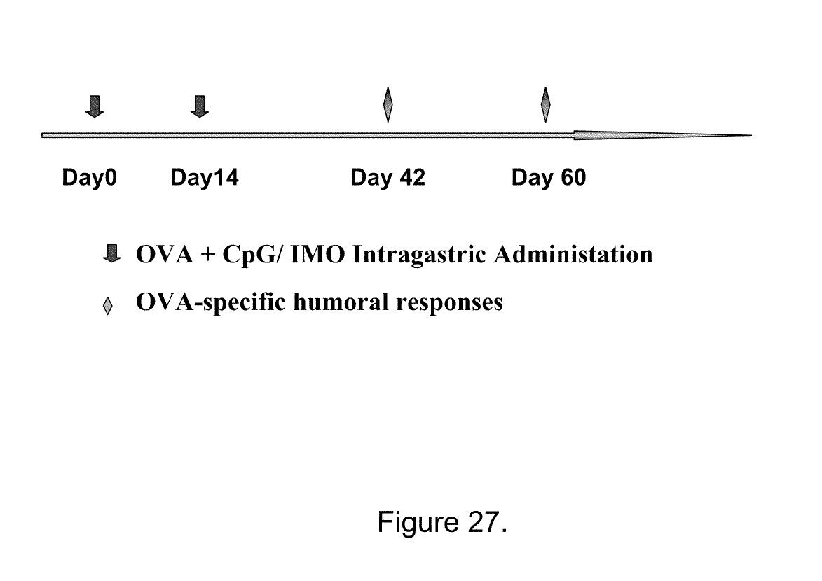

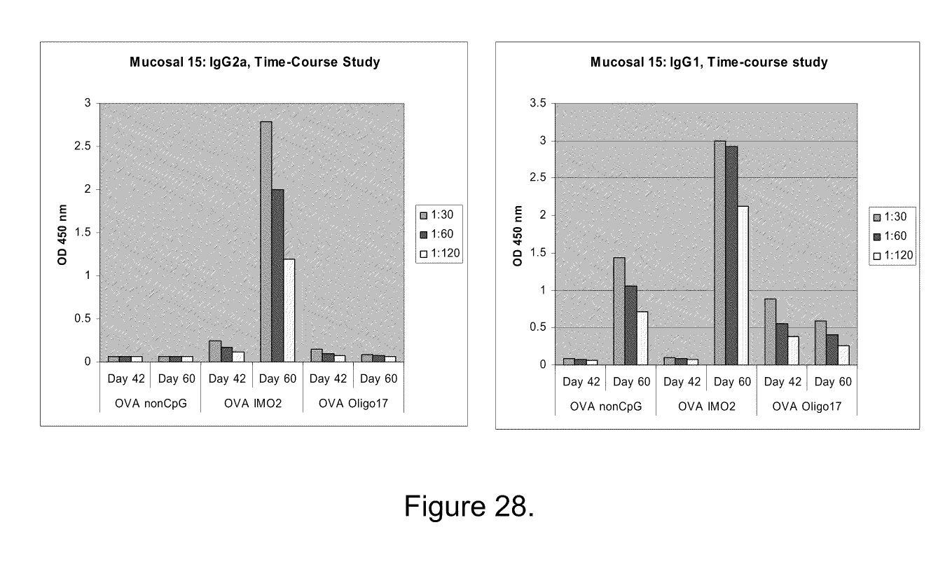

As shown in FIG. 27, C57BL/6 mice were administrated i.g with 5 mg/kg OVA only or mixed with 5 mg/kg linear CpG oligo (1182) or IMO 2048 on days 1 and 14 and OVA-specific serum antibody responses were evaluated at day 42 and day 60 by ELISA. As shown in FIG. 28, the level of OVA-specific IgG2a at day 42 started to increase in OVA-2048 group. Meanwhile, OVA-specific IgG1 was completely suppressed in OVA-2048 group at this time point. By day 60, anti-OVA IgG2a continued to rise in OVA-2048 group and OVA-specific IgG2a in OVA-1182 group decreased to the OVA-2092 (non CpG DNA oligonucleotide) control group level. Meanwhile, anti-OVA IgG1 titer dramatically increase at this time point, suggesting persisting presence of OVA-2048 is needed for remaining Th1 dominating status in OVA-2048 group.

### Example 22

### IMO-Mediated Intragastric Vaccination Immunization Schedule Study

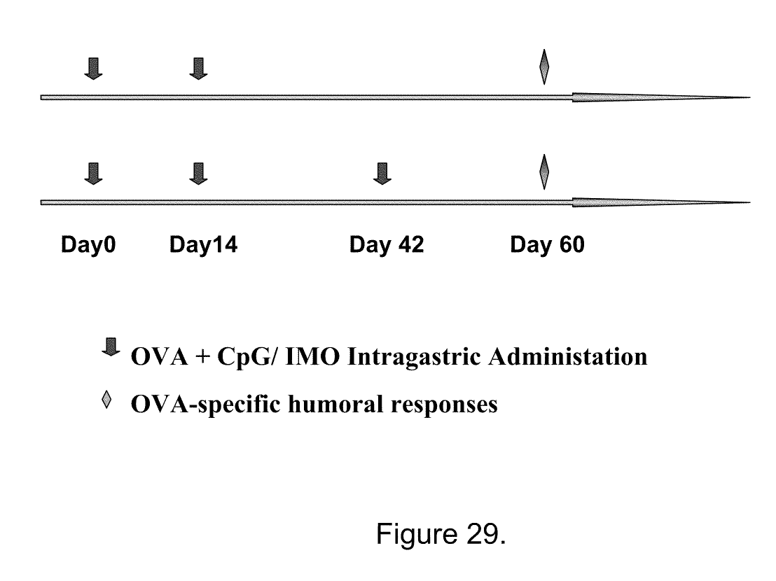

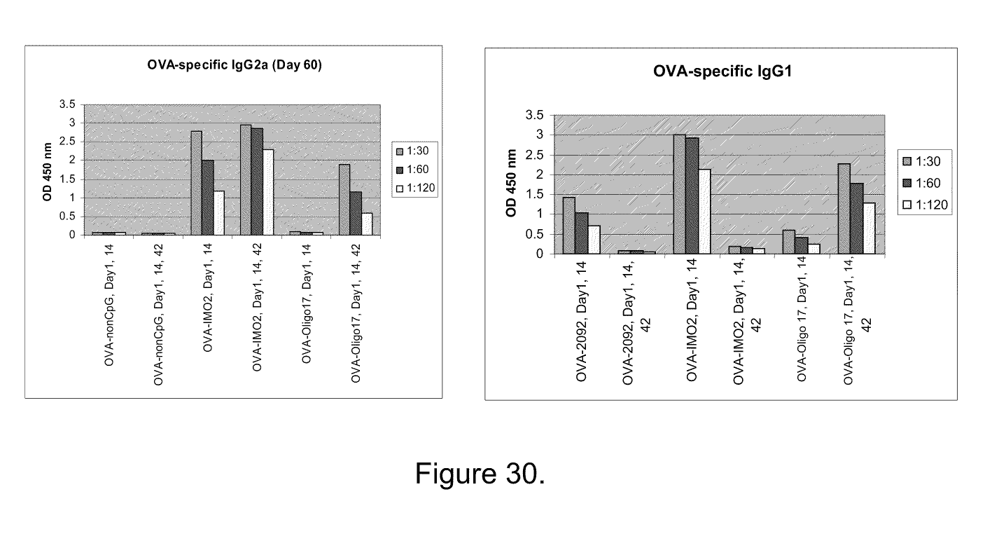

As shown in FIG. 29, C57BL/6 mice were administrated i.g with 5 mg/kg OVA only or mixed with 5 mg/kg 1182 or 2048 on days 1 and 14 (Group 1) or Days 1, 14 and 42 (Group 2). OVA-specific serum antibody responses were evaluated at day 42 and day 60 by ELISA. FIG. 30 shows that persistent presence of OVA-2048 is needed for maintaining of Th1 dominated statue in OVA-2048 group. In two immunization group (Days 1 and 14), there were both high titers of OVA-specific IgG2a and IgG1 in OVA-2048 group at day 60, suggesting immune responses shifted from Th1 type at early time point (day 42) to both Th1 and Th2 mixture at later time point (Day 60). The humoral response elicited by OVA-1182 i.g immunizations lasted significantly shorter as IgG2a decreased to control group levels at day 60. Further immunization at day 42 can reverse such responses, as it significantly inhibited OVA-specific IgG1 in OVA-2048 group.

### Example 23

### IMO-Mediated Intragastric Vaccination Dose-Dependent Responses

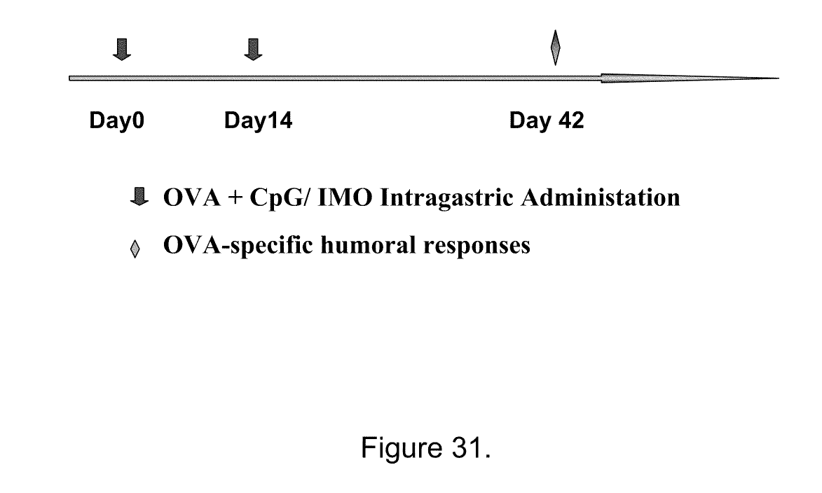

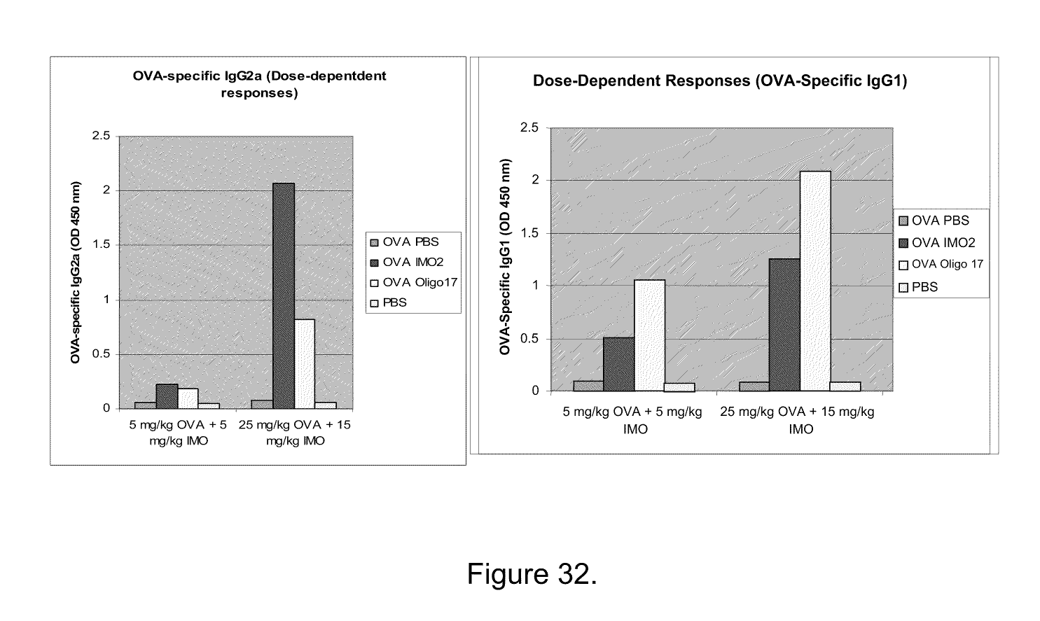

As shown in FIG. 31, C57BL/6 mice were administrated i.g with 5 mg/kg or 25 mg/kg OVA only, or mixed with 5 mg/kg or 15 mg/kg linear CpG oligo (1182) or IMO 2048 on days 1 and 14, and OVA-specific serum antibody responses were evaluated at day 42 by ELISA. FIG. 32 shows that OVA-2048 induced stronger Th1-type responses.

### Example 24

### IMO-Mediated Intragastric Vaccination Tumor Challenge Study

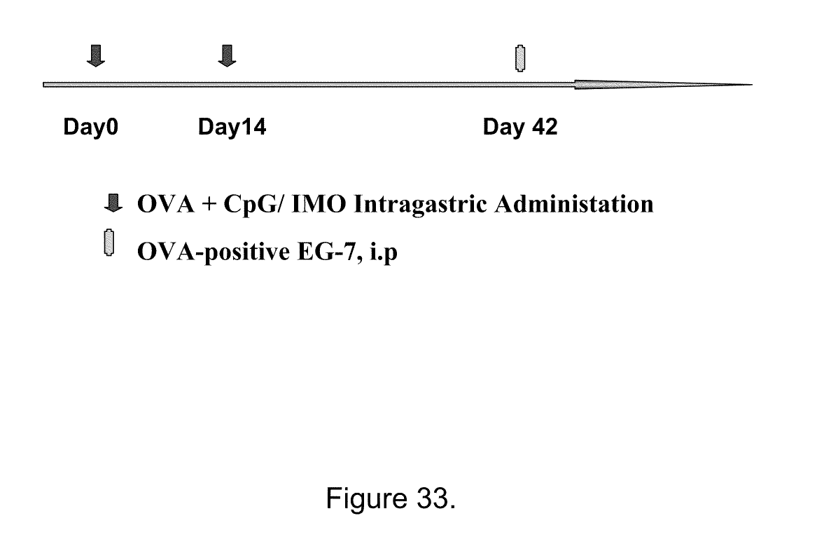

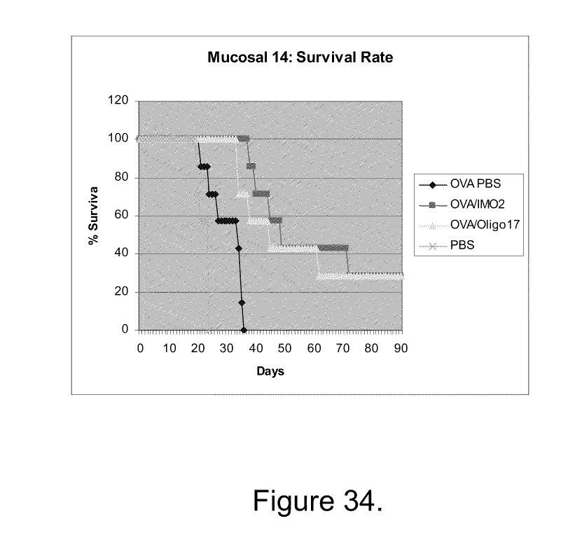

As shown in FIG. 33, C57BL/6 (n=10) mice were i.g administrated with 25 mg/kg OVA mixed with 15 mg/kg 2048 or 1182 in 400 ml PBS on days 1 and 14 and the immunized mice (n=7) were i.p challenged with 1.5×10⁶ OVA-positive EG-7 cells on day 42. FIG. 34 shows that both OVA-1182 and OVA-2048 elicited antigen-specific tumor rejection, however, different immune response profiles were elicited by OVA in 2048 or 1182 and may result in different immune protection against OVA positive EG-7 tumor cell challenge.

While the foregoing invention has been described in some detail for purposes of clarity and understanding, it will be appreciated by one skilled in the art from a reading of this disclosure that various changes in form and detail can be made without departing from the true scope of the invention and appended claims.

We claim:

1. A method for preventing a disease or disorder in a mammal, the method comprising administering an immunomer to the mucosal lining of the mammal, wherein the immunomer comprises at least two oligonucleotides linked by a non-nucleotidic linker and having more than one 5′ end, wherein at least one of the oligonucleotides is an immunostimulatory oligonucleotide having an accessible 5′ end and comprises an immunostimulatory dinucleotide selected from the group consisting of C*pG, CpG*, and C*pG*, wherein C is cytidine or 2′-deoxycytidine, C* is 2′-deoxythymidine, arabinocytidine, 1-(2′-deoxy-β-D-ribofuranosyl)-2-oxo-7-deaza-8-methyl-purine, 2′-deoxy-2′-substituted-arabinocytidine, 2′-O-substituted-arabinocytidine, 2′-deoxy-5-hydroxycytidine, 2′-deoxy-N4-alkyl-cytidine, 2′-deoxy-4-thiouridine or other non-natural pyrimidine nucleoside, G is guanosine or 2′-deoxyguanosine, G* is 2′-deoxy-7-deazaguanosine, 2′-deoxy-6-thioguanosine, arabinoguanosine, 2′-deoxyinosine, 2′-deoxy-2′ substituted-arabinoguanosine, 2′-O-substituted-arabinoguanosine, wherein the route of administration is selected from the group consisting of oral, intratracheal, intrarectal, intravaginal and intragastric administration.

2. The method according to claim 1, wherein the mammal is selected from the group consisting of rats, mice, cats, dogs, horses, cattle, cows, pigs, rabbits, non-human primates, and humans.

3. The method according to claim 1, further comprising administering an agent selected from the group consisting of vaccines, allergens, antigens, antibodies, monoclonal antibodies, chemotherapeutic drugs, antibiotics, lipids, DNA vaccines and other adjuvants such as alum.

4. The method according to claim 3, wherein the immunomer or the antigen, or both, are linked to an immunogenic protein or non-immunogenic protein.

5. The method according to claim 1, further comprising administering an adjuvant.
